(19) United States
(12) Patent Application Publication
(10) Pub. No.: US 2009/0143808 A1
(43) Pub. Date: Jun. 4, 2009

(54) Guided Tissue Cutting Device, Method of Use and Kits Therefor

(76) Inventor: Russell A. Houser, Livermore, CA (US)

Correspondence Address: WILSON SONSINI GOODRICH & ROSATI, 650 PAGE MILL ROAD, PALO ALTO, CA, 94304-1050, US

(21) Appl. No.: 12/327,655

(22) Filed: Dec. 3, 2008

Related U.S. Application Data
(63) Continuation-in-part of application No. 12/263,322, filed on Oct. 31, 2008.
Application No. 12/263,322 is a continuation-in-part of application No. 10/183,396, filed on Jun. 28, 2002, now Pat. No. 6,726,696.
Application No. 10/183,396 is a continuation-in-part of application No. 10/127,714, filed on Apr. 23, 2002, now abandoned.
(60) Provisional application No. 60/286,269, filed on Apr. 24, 2001.
(60) Provisional application No. 60/300,892, filed on Jun. 25, 2001.
(60) Provisional application No. 60/302,255, filed on Jun. 28, 2001.

(51) Int. Cl. A61B 17/32; A61B 18/04

(52) U.S. Cl. 606/170; 606/172; 606/27

(57) ABSTRACT

The present invention relates to a guided tissue cutter configured to receive a cutter adapted to cut a target, and an elongate channel along the length of the tissue cutter adaptable to slide or rotate along or around an elongate member, The elongate member can be a medical device. The tissue cutter can comprise one or more stop members that permit the cutter to penetrate a predetermined region of a target tissue to a predetermined depth and to inhibit further penetration of a tissue beyond the predetermined region of the target tissue. The tissue cutter can be configured to clip or snap on and off of the elongate member.

### RELATED APPLICATIONS

This application is a continuation-in-part of and claims priority to U.S. patent application Ser. Nos. 10/127,714, filed Apr. 23, 2002 and entitled “Arteriotomy Closure Devices and Techniques,” and 12/263,322, filed Oct. 31, 2008 and titled “Vascular Closure Devices, Systems, and Methods of Use” and claims the benefit of priority from U.S. Provisional Patent Application Nos. 60/286,269, filed Apr. 24, 2001 and entitled “Percutaneous Vessel Access Closure Device and Method,” 60/300,892, filed Jun. 25, 2001 and entitled “Percutaneous Vessel Access Closure Device and Method,” 60/302,255, filed Jun. 28, 2001 and entitled “Percutaneous Vessel Access Closure Device and Method (Hemostatic Patch or Collar),” 61/005,435, filed Dec. 3, 2007 and entitled “Guided Tissue Cutting Device and Method of Use,” 61/190,100, filed Aug. 26, 2008 and entitled “Tissue Closure Devices, Systems and Methods of Use,” the disclosures of which are hereby incorporated by reference herein in their entirety and made a part of the present specification.

### BACKGROUND OF THE INVENTION

I. Field of the Invention

The invention generally relates to medical devices and techniques, and more particularly to cardiovascular tissue closure devices and techniques.

II. Description of the Related Art

In most cardiology and radiology procedures, a catheter is inserted into an artery, such as the femoral artery, through a vascular introducer. When the procedure is complete, the physician removes the catheter from the introducer and then removes the introducer from the arteriotomy in the vessel. The physician then must prevent or limit the amount of blood that leaks through the arteriotomy so that the patient can be discharged. Physicians currently use a number of methods to close the arteriotomy, such as localized compression, sutures, collagen plugs, adhesives, gels, foams, clips, and similar materials.

In performing localized compression, the physician presses down against the vessel to allow the arteriotomy to naturally clot. This method, however, can take a significant amount of time, and requires the patient to remain immobilized and kept in the hospital for observation. Moreover, clots at the puncture site may also be dislodged. The amount of time necessary for the compression can significantly increase depending upon how much heparin, glycoprotein IIb/IIA antagonists, or other anti-clotting agents were used during the procedure. Sutures and collagen plugs can have procedure variability, can require time to close the vessel, and can necessitate a separate deployment device. Adhesives, gels, foams, and clips can have negative cost factors, can necessitate a complicated deployment process, and can have procedure variability.

### SUMMARY OF THE INVENTION

A tissue closure system can include a deployment instrument and a sealing element. The deployment instrument can be slidably mounted to and guided by a tubular, or substantially tubular, or non-tubular, medical device. The deployment instrument can be advanced over the medical device to the desired location. The sealing element can then be advanced off of the end of the tool. The sealing element can include tissue engaging elements that are configured to automatically close upon deployment to bring together tissue. A slidably attached guided skin (or other tissue) cutter can also be used if desired to facilitate entry of the deployment instrument.

A clip for closing an opening in a blood vessel can include a base with a substantially circular, substantially continuous upper edge, the base having substantially the same shape and orientation in both a pre-deployed and deployed state, a plurality of fingers extending from a distal edge of the base, the fingers including a window and a flexion region configured to facilitate bending and one or more tines. The tines can be generally perpendicular to the base in the pre-deployed state and can be canted inwardly toward each other in the deployed state. The tines can be configured to close a blood vessel opening from an external wall of the vessel.

In certain embodiments, at least two fingers can each have a different number of tines. The tines of at least two of the fingers can be interlaced with each other in the deployed state. The tines can be substantially pointed. The size of the tines can be configured so that the depth of penetration into a wall of the vessel in the deployed state is less than the thickness of the vessel wall. The clip can include a retrieval connection for removing the clip from a patient after substantial hemostasis is achieved.

A deployment device for deploying a vessel closure clip can include a first inner tubular member and a second outer tubular member, the inner and outer tubular members being adaptable to be slideably engaged such that the inner tubular member and the outer tubular move longitudinally with respect to each other along at least a portion of an elongate medical device, a clip-receiving region located at a distal region of the deployment device, and a clip retention structure configured to maintain a clip in a pre-deployed state during advancement of the deployment device percutaneously toward a wall of the vessel. In certain embodiments, the deployment device can be configured such that longitudinal movement between the inner and outer tubular members of the deployment device transitions the clip from a first, pre-deployed state to a second, partial deployment state after contacting a vessel wall. The deployment device can be configured such that further relative longitudinal movement between the inner and outer tubular members produces a second, full deployment state and releases the clip from the deployment device.

In certain embodiments, the deployment device can include a pressure responsive element for providing feedback regarding the force applied by the deployment device against a vessel wall. The deployment device can be configured such that one or more medical implements can be passed through the deployment device in the partial deployment state. The deployment device can be configured such that introduction of foreign material into the interior of the blood vessel is not required in order to close an opening in the vessel. The deployment device can include a stop element configured to permit relative longitudinal movement between the inner and outer tubular members of a predetermined distance and further configured to inhibit further relative longitudinal movement in a first direction, the predetermined distance being sufficient to transition the clip to the second, partial deployment state without releasing the clip from the deployment device. The deployment device can include a releasing element configured to permit the stop element to be overcome to allow further relative longitudinal movement between the inner and outer tubular members in the first direction.

A system for closing an opening in a blood vessel can include a deployment device and a vascular closure clip. In certain embodiments, the system can also include a slidable tissue cutter. The deployment device can include a first inner tubular member and a second outer tubular member, the inner and outer tubular members being adapted to move longitudinally with respect to each other along at least a portion of an elongate medical device. The deployment device can further include a clip-receiving region located at a distal region of the deployment device and a clip retention structure configured to maintain a clip in a pre-deployed state during advancement of the deployment device percutaneously toward a wall of the vessel. The deployment device can be configured such that relative longitudinal movement between the inner and outer tubular members transitions the clip from a first, pre-deployed state to a second, partial deployment state after contacting a vessel wall. The deployment device can be configured such that further relative longitudinal movement between the inner and outer tubular members produces a second, full deployment state and releases the clip from the deployment device. The vascular closure clip can include a base and a plurality of fingers extending from an edge of the base, the fingers including one or more tines. The tines can be generally perpendicular to the base in the pre-deployed state and canted inwardly toward each other in the deployed state. The clip can be biased in the deployed state. The tines can be configured to close a blood vessel opening from an external wall of the vessel.

A method of deploying a vascular closure device can include the steps of: providing a deployment device with a clip in a pre-deployed state loaded thereon; advancing a distal end of the deployment device to an opening in a blood vessel such that a portion of the clip extends into an exterior wall of the vessel; producing generally longitudinal relative movement between an inner and outer tubular member of the deployment device to transition the clip from the pre-deployed state to a partially deployed state in which one or more previously used medical implements can be passed through the clip and removed from the patient; and producing further generally longitudinal relative movement between the inner and outer tubular members to transition the clip from the partially deployed state to a fully deployed state in which opposing sides of the opening in the vessel wall are pulled toward each other to close the opening and the clip is released from the deployment device. In certain embodiments, the method of deployment a vascular closure device can also include removing the clip from the vessel wall after substantial hemostasis is achieved.

A tissue cutter for use in a vascular closure procedure can include a handle portion, an attachment portion configured to removably attach the cutter to an elongate member, and a static cutting portion configured to cut a predetermined region of tissue near an opening through which the elongate member has been inserted to increase the size of the opening as the tissue cutter is advanced toward the opening.

In certain embodiments, the handle portions of the tissue cutter can flare outwardly at a proximal region of the handle portions. The tissue cutter can include a stop member configured to permit the static cutting portion to penetrate the predetermined region of tissue to a predetermined depth and to inhibit further penetration of the predetermined region of tissue. The static cutting portion can be configured to increase the size of the opening sufficiently to permit percutaneous insertion of a deployment device.

A method of increasing the size of a tissue opening to facilitate percutaneous insertion of a medical device can include the steps of: providing a tissue cutter including a handle portion, a static cutting portion, and a stop portion; removably attaching the cutter to an elongate member that has been inserted percutaneously into an opening in a patient; advancing the cutter along the elongate member toward the patient; engaging the cutting portion with a predetermined region of tissue near the opening in the patient to increase the size of the opening; further advancing the cutter against the tissue until the stop member engages the predetermined region of tissue, the stop member permitting the cutting portion to penetrate the predetermined region of tissue to a predetermined depth and thereafter inhibiting further penetration of the predetermined region of tissue; and removing the cutter from the elongate member.

A system for closing an opening in a blood vessel can include a deployment device and a plug. The deployment device can include an inner tubular member and an outer tubular member, the inner tubular member received within an inner lumen of the outer tubular member, the inner and outer tubular members being adapted to move longitudinally with respect to each other, an inner lumen of the inner tubular member configured to receive an elongate medical device, the deployment device being configured to be advanced longitudinally over the elongate medical device. The deployment device can also include a plug receiving region located at a distal end of the deployment device and configured to receive a plug. The deployment device can be configured such that relative longitudinal movement between the inner and outer tubular members releases the plug from the deployment device. The plug can include a first portion having a first cross-sectional areas the first portion being configured to be received within the plug receiving region. The plug can also include a second portion having a second cross-sectional area, the second cross-sectional area being larger than the first cross-sectional area, the second portion being sized so as to be larger than an opening in a vessel when the plug is delivered to the vessel opening. The plug can also include a longitudinal channel passing through the first and second portions, the longitudinal channel being configured to receive the elongate medical device. The plug can include a swellable material configured to swell when exposed to a fluid to thereby substantially occlude the longitudinal channel. The second portion can be configured to be received against an outer wall of the vessel. The second portion can be configured to act as a stop to prevent overinsertion of the plug.

A method of deploying a vascular closure device can include the steps of: providing a deployment device with a plug in a non-swelled state loaded thereon, the plug including a swellable material that swells when exposed to fluid, the plug further including a first portion having a first cross-sectional area and a second portion having a second cross-sectional area, the second cross-sectional area being larger than the first cross-sectional area, the plug having a longitudinal channel passing through the first and second portions; advancing a distal end of the deployment device over an elongate medical device to an opening in a blood vessel such that the second portion is received against an outer wall of the vessel opening and the plug is exposed to a bodily fluid; and producing generally longitudinal relative movement between an inner and outer tubular member of the deployment device to release the plug from the deployment device; wherein the plug swells upon the exposure to the bodily fluid to substantially occlude the longitudinal channel.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIG. 1 shows a perspective view of an embodiment of a vessel closure system;

FIG. 2 shows a perspective view of an embodiment of a vascular closure clip in an open or pre-deployed configuration;

FIG. 3 shows a perspective view of the clip of FIG. 2 in a closed or deployed configuration;

FIG. 4 shows a side view of the clip of FIG. 2 in an open configuration;

FIG. 5 shows a side view of the clip of FIG. 2 in a closed configuration;

FIG. 6 shows a bottom view of the clip of FIG. 2 in a closed configuration;

FIG. 7 shows a perspective view of a deployment instrument preloaded with a vascular closure clip;

FIG. 8 shows a close-up view of the distal end of the deployment instrument of FIG. 7;

FIG. 9 shows a perspective view of an inner tubular member portion of the deployment instrument of FIG. 7;

FIG. 10 shows a side view of the inner tubular member of FIG. 9;

FIG. 11 shows a distal end view of the inner tubular member of FIG. 9;

FIG. 12 shows a perspective view of an outer tubular member portion of the deployment instrument of FIG. 7;

FIG. 13 shows a distal end view of the outer tubular member of FIG. 12;

FIG. 14 shows a side view of the outer tubular member of FIG. 12;

FIG. 15 shows a close-up side view of an intermediate portion of the outer tubular member of FIG. 12;

FIG. 16 shows another close-up side view of an intermediate portion of the outer tubular member of FIG. 12;

FIG. 17 shows a perspective view of a pressure element of the deployment instrument of FIG. 7;

FIG. 18 shows a perspective view of the deployment instrument of FIG. 7 loaded onto a vascular introducer that has been inserted into a patients blood vessel;

FIG. 19 shows a perspective view of the deployment instrument of FIG. 7 which has been advanced over the vascular introducer until its distal end encounters the vessel wall;

FIG. 20 shows a close-up view of the deployment instrument of FIG. 19 showing the pressure element in an initial, relaxed position;

FIG. 21 shows a perspective view of the deployment instrument of FIG. 7 with the pressure element fully advanced;

FIG. 22 shows a close-up view of the deployment instrument of FIG. 21 showing the fully advanced pressure element;

FIG. 23 shows a close-up side view of the deployment instrument of FIG. 7 in a partially-deployed state showing the clip's tines penetrating the vessel wall;

FIG. 24 shows a perspective view of the deployment instrument of FIG. 7 in a partially-deployed state;

FIG. 25 shows a close-up view of the distal end of the deployment instrument of FIG. 24;

FIG. 26 shows a close-up bottom view of the proximal end of the deployment instrument of FIG. 24 showing the handle engaging the stop element;

FIG. 27 shows a side view of the proximal end of the deployment instrument of FIG. 24 showing the handle engaging the stop element;

FIG. 28 shows a side view of the deployment instrument of FIG. 24 in a partially deployed state after withdrawing the vascular introducer;

FIG. 29 shows a side view of the proximal end of the deployment instrument of FIG. 27 showing how the stop element can be overcome;

FIG. 30 shows a perspective view of the deployment instrument of FIG. 7 in a fully deployed configuration;

FIG. 31 shows a side view of the deployment instrument of FIG. 7 in a fully deployed configuration which shows the vascular closure clip closing the arteriotomy;

FIG. 32 shows a side view of the deployment instrument of FIG. 7 showing the deployment instrument being removed from the patient's body following deployment;

FIG. 33 shows a side view of a vascular closure procedure using a removable clip, showing the deployment instrument being advanced over the vascular introducer;

FIG. 34 shows a side view of the procedure of FIG. 33 showing the deployment instrument being removed after deploying the clip;

FIG. 35 shows a side view of the vascular closure procedure of FIG. 33 showing the vascular closure clip being removed from the patient's body following hemostasis;

FIG. 36 shows a perspective view of a clip loading mechanism;

FIG. 37 shows a perspective view of the clip loading mechanism of FIG. 36 fully inserted into the distal end of the deployment instrument;

FIG. 38 shows a perspective view of a pusher tool configured to mate with a vascular closure clip to fully advance the clip over the clip loading mechanism of FIG. 36 and onto the distal end of the deployment instrument;

FIG. 39 shows a perspective view of the pusher tool of FIG. 38 fully advancing the clip onto the distal end of the deployment instrument;

FIG. 40 shows a bottom view of a slidable tissue cutter;

FIG. 41 shows a perspective view of the slidable tissue cutter of FIG. 40;

FIG. 42 shows a bottom view of a frame which can constitute a first component of the slidable tissue cutter of FIG. 40;

FIG. 43 shows a distal end view of the frame of FIG. 42;

FIG. 44 shows a perspective view of a slidable tissue dilator;

FIG. 45 shows a distal end view of the slidable tissue dilator of FIG. 44;

FIG. 46 shows a side view of the slidable tissue dilator of FIG. 44;

FIG. 47A shows a perspective view of another embodiment of a vascular closure clip in an open configuration;

FIG. 47B shows a perspective view of the vascular closure clip of FIG. 47A in a closed configuration;

FIG. 47C shows a bottom view of the vascular closure clip of FIG. 47A in a closed configuration;

FIG. 47D shows a side view of the vascular closure clip of FIG. 47A in a closed configuration;

FIG. 48A shows a perspective view of another embodiment of a vascular closure clip in a closed configuration;

FIG. 48B shows a perspective view of the vascular closure clip of FIG. 48A in an open configuration;

FIG. 49A shows a perspective view of another embodiment of a vascular closure clip in an open configuration;

FIG. 49B shows a perspective view of the vascular closure clip of FIG. 49B in a closed configuration;

FIG. 50A shows a perspective view of another embodiment of a vascular closure clip in an open configuration;

FIG. 50B shows a perspective view of the vascular closure clip of FIG. 50A in a closed configuration;

FIG. 51A shows a perspective view of another embodiment of a vascular closure clip in an open configuration;

FIG. 51B shows a perspective view of the vascular closure clip of FIG. 51A in a closed configuration;

FIG. 51C shows a side view of the vascular closure clip of FIG. 51A in an open configuration;

FIG. 51D shows a side view of the vascular closure clip of FIG. 51A in a closed configuration;

FIG. 51E shows a top view of the vascular closure clip of FIG. 51A in a closed configuration;

FIG. 52 shows a circuit diagram of a circuit using direct resistive element heating to heat tissue surrounding the arteriotomy;

FIG. 53 shows a circuit diagram of a circuit using ohmic tissue beating to heat tissue surrounding the arteriotomy;

FIG. 54 shows a distal end view of another embodiment of an inner tubular member that can form one component of a deployment instrument;

FIG. 55 shows a proximal end view of the inner tubular member of FIG. 54;

FIG. 56A shows a perspective view another embodiment of a deployment instrument which can be used with a vascular closure plug;

FIG. 56B shows a perspective view of the deployment instrument of FIG. 56A preloaded with a vascular closure plug;

FIG. 56C shows a perspective view of the deployment instrument of FIG. 56A after deploying the vascular closure plug;

FIG. 57 shows a side view of the deployment instrument of FIG. 56B being advanced over a vascular introducer that has been inserted into a patients blood vessel;

FIG. 58 shows a side view of the deployment instrument of FIG. 57 positioning the distal end of the vascular closure plug against the arteriotomy;

FIG. 59 shows a side view of the deployment instrument of FIG. 57 holding the plug against the arteriotomy after removing the vascular introducer;

FIG. 60 shows a side view of the deployment instrument of FIG. 57 showing the exposed portions of the plug beginning to swell;

FIG. 61 shows a side view of a deployed plug as the deployment instrument of FIG. 57 is removed;

FIG. 62 shows a side view of the deployed plug of FIG. 61 which is continuing to swell; and

FIG. 63 shows a side view of the deployed plug of FIG. 61 which has begun to be absorbed by the patient's body.

### DETAILED DESCRIPTION

The following description provides examples of certain embodiments for purposes of illustration. The inventions as claimed should not be limited to these examples. Moreover, although the examples are provided in the context of vessel closure, the invention also has broad application to other types of tissue closure. U.S. Pat. No. 7,025,776 to Houser et al., the entirety of which is incorporated herein by reference, discloses a variety of additional vessel closure devices and methods with features that can be used in combination with or instead of features of the embodiments disclosed herein.

I. Vessel Closure System

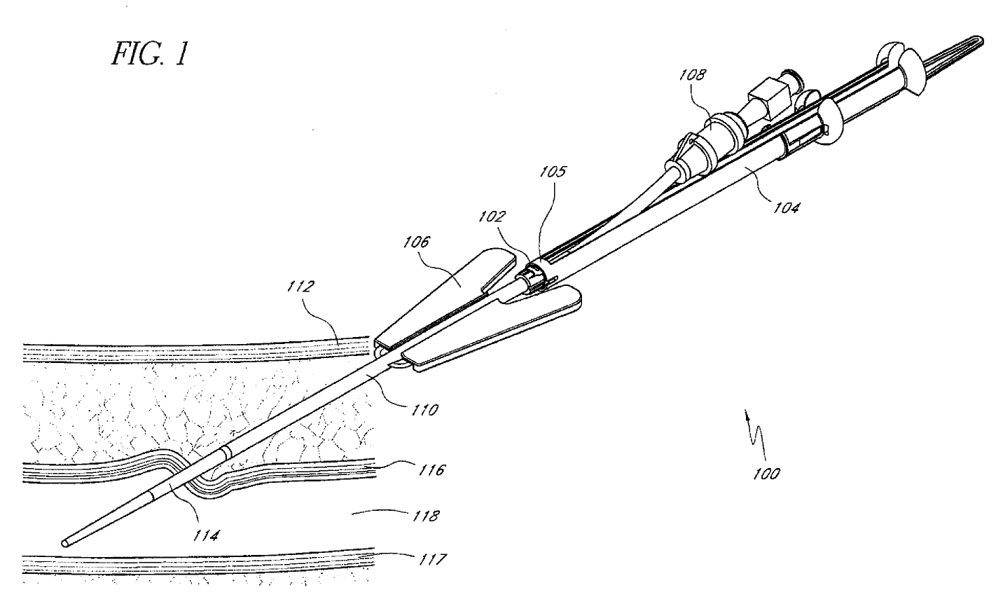

Referring to FIG. 1, a vessel closure system 100 can include a vessel closure device such as clip 102 and a deployment or advancement instrument 104. Clip 102 is loaded onto a distal end 105 of deployment instrument 104. The deployment instrument 104 is slidably mounted to or advanced along and generally guided by a vascular introducer 108 or other tubular medical device such as a catheter which has been inserted into a blood vessel 118. In certain embodiments, a narrow opening in the skin initially created for the insertion of the vascular introducer 108 can be expanded or enlarged by a guided slidable tissue cutter 106 to form a percutaneous opening 112 sufficiently large to easily permit passage of the deployment instrument 104 into the body.

The deployment instrument 104 can be guided by a tube section 110 of vascular introducer 108 through the percutaneous opening 112 until it reaches arteriotomy site 114. The deployment instrument 104 is configured to deploy a vascular closure clip 102 to close the arteriotomy 114. The deployment instrument 104 can then be withdrawn.
(a) Clips

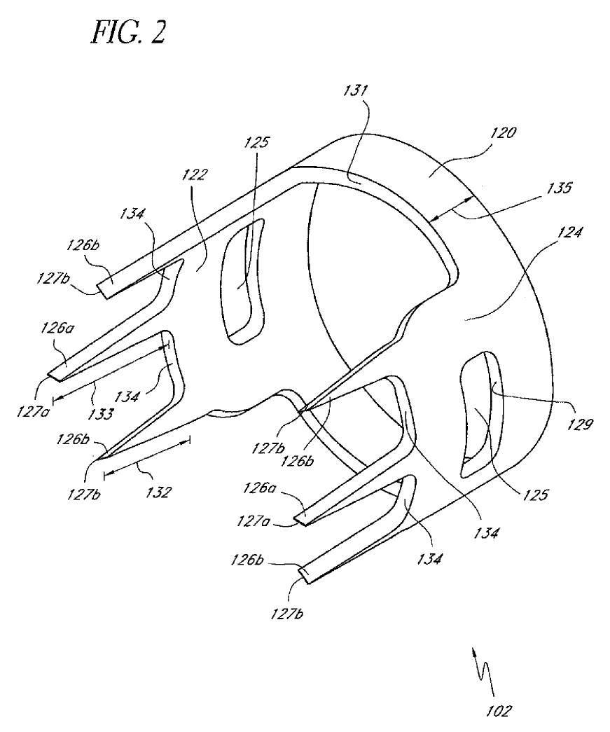

FIG. 2 is a perspective view of an embodiment of a clip 102 in a pre-deployed or open configuration. Clip 102 can include a base portion 120. Base portion 120 can be generally or completely annular, forming a partial or complete circle. In some embodiments, a base portion 120 with a continuous or substantially continuous circle along its upper edge as illustrated can provide increased strength and resistance to contortion or bending in either or both of the open and closed configurations. A generally circular base portion 120 can allow the tines 126a-b to rove or bend during the transition between the open and closed configurations while generally resisting a substantial change in shape or orientation of the base portion 120. The height 135 of the base portion 120 can be selected to achieve a desired amount of stiffness or flexibility.

Fingers 122 and 124 can be configurable to extend from base portion 20 and support a plurality of tissue-engaging elements such as tines 126a-b. In some embodiments, as illustrated, the fingers 122 and 124 can be positioned in a substantially opposing arrangement, for example wherein finger 122 is positioned in a substantially diametrically opposite location on the generally circular base 120 from finger 124. As explained below, many other positions and configurations can be used.

In the illustrated example of FIG. 2, each finger 122, 124 includes three tines: one central tine 126a and two outer tines 126b. The outer tines 126b can be substantially the same length 132 from the respective tips 127b to the respective junctures with the forward surface 134 of each finger 122, 124. In some embodiments, the forward surface 134 can be substantially perpendicular to the tines 126a, 126b and substantially parallel with the plane of the base 120 in the open configuration. Surfaces 134 can generally act as substantially blunt stops to prevent over-insertion of clip 102 into the vessel wall 116. In some embodiments, the length 133 of the central tines 126a can be slightly greater than the length 132 of the outer tines 126b. This length differential can assist in producing an increased leverage and an increased force along a central line generally bisecting the base 120 between the two opposing central tines 126a to help pull generally opposing sides of a tissue slit opening together.

In some embodiments, the lengths 132, 133 can be selected so that the tines 126a, 126b pierce but do not completely penetrate through a vessel wall 116 of average thickness into the interior region of the vessel 118. For example, the length 132 may be greater than or equal to about 1 mm, and/or the length 132 may be less than or equal to about 4 mm and the length 133 may be greater than or equal to about 1 mm, and/or the length 133 may be less than or equal to about 5 mm. In some embodiments, the length 132 is about 3 mm, and the length 133 is about 3 mm. In other embodiments, the tines 126a, 126b can be configured to penetrate the vessel wall, but generally not long enough to contact or penetrate the vessel wall 117 on the opposite side of the vessel 118. The lengths of the tines 126a, 126b are generally greater than the height 135 of the base portion 120. In the illustrated embodiment, fingers 122 and 124 are generally symmetrical about a central axis. In other embodiments, the fingers 122, 124 can be asymmetrical or include a different number or configuration of tissue-engaging elements.

Fingers 122, 124 can include one or more bend-facilitating regions 125, such as narrowed regions, indentations, articulating joints, or window portions as illustrated. The size, shape, and placement of the bend-facilitating regions 125 can be adjusted to assist in achieving a desired amount of closure force for the clip 102. As illustrated, the contours of the bend-facilitating regions 125 can be generally smooth to avoid additional trauma to the vessel wall. In some embodiments, an upper edge 129 of a bend-facilitating region 125 can be positioned in general alignment with a lower edge 131 of the base portion 120 to maintain a desired height 135 of the base portion 120. As illustrated, the width of the bend-facilitating region can be smaller than the height 135 of the base portion 120.

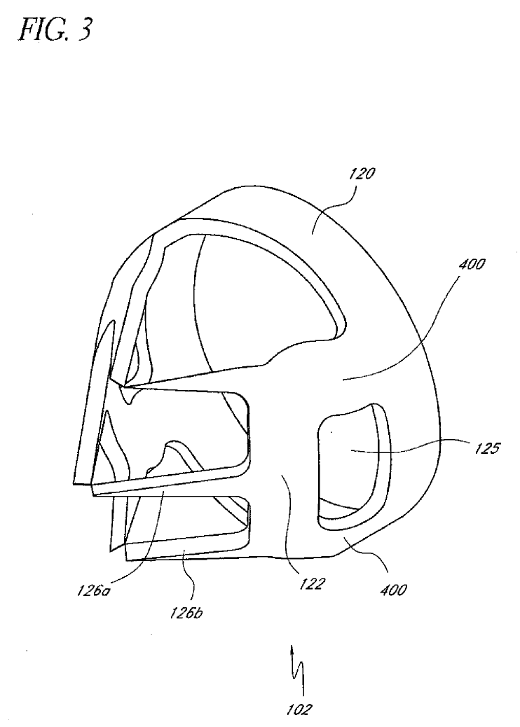

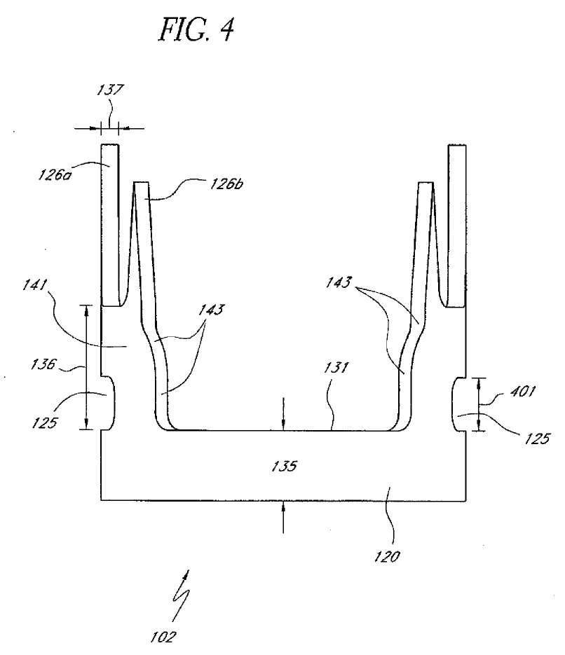

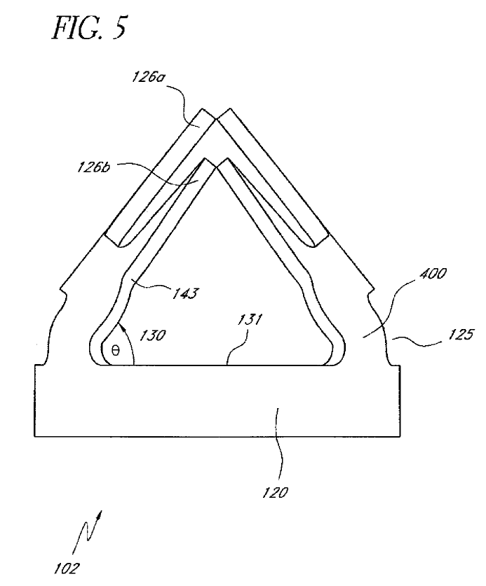

FIG. 3 shows a perspective view of clip 102 in a closed or deployed configuration. Clip 102 is preferably biased into a closed configuration. As shown in FIGS. 1 and 2, clip 102 can be temporarily maintained in an open or pre-deployed state by deployment instrument 104 until it is deployed and returns to substantially the same configuration illustrated in FIG. 3. Clip 102 can be configured to automatically close upon deployment to close the arteriotomy. In certain embodiments, the closing of clip 102 can be accomplished substantially via changes in flexion regions 400. In some embodiments, the dimensions, shape, and/or orientation of other portions of clip 102 can remain substantially unchanged between the pre-deployed and deployed states.

FIG. 4 is a side view of clip 102 in an open configuration. The respective heights 135, 136, 401, of the base portion 120, the support portion 141, and the bend-facilitating region 125, can have many different values, depending on the particular application of the clip 102 and other design preferences. Moreover, these heights 135, 136, 401 can be constant or can vary in some embodiments. By way of example, the height 135 of the base portion 120 may be greater than or equal to about 0.5 mm and/or may be less than or equal to about 2 mm; the height 136 of the support portion 141 may be greater than or equal to about 0.5 mm and/or may be less than or equal to about 4 mm; and the height 401 of the bend-facilitating region 125 may be greater than or equal to about 0.2 mm and/or may be less than or equal to about 2 mm. In some embodiments, the height 135 of the base portion 120 is about 0.5 mm, 0.6 mm, 0.7 mm, 0.8 mm, 0.9 mm, 1.0 mm, 1.1 mm, 1.2 mm, 1.3 mm, 1.4 mm, 1.5 mm, 1.6 mm, 1.7 mm, 1.8 mm, 1.9 mm or 2.0 mm. In some embodiments, the height 136 of the support portion 141 is about 0.5 mm, 0.6 mm, 0.7 mm, 0.8 mm, 0.9 mm, 1.0 mm, 1.1 mm, 1.2 mm, 1.3 mm, 1.4 mm, 1.5 mm, 1.6 mm, 1.7 mm, 1.8 mm, 1.9 mm, 2.0 mm, 2.1 m 2.2 mm, 2.3 mm, 2.4 mm, 2.5 mm, 2.6 mm, 2.7 mm, 2.8 mm, 2.9 mm, 3.0 mm, 3.1 mm, 3.2 mm, 3.3 mm, 3.4 mm, 3.5 mm, 3.6 mm, 3.7 mm, 3.8 mm, 3.9 mm, or 4.0 mm. In some embodiments, the height 401 of the bend-facilitating region 125 is about 0.2 mm, 0.3 mm, 0.4 mm, 0.5 mm, 0.6 mm, 0.7 mm, 0.8 mm, 0.9 mm, 1.0 mm, 1.1 mm, 1.2 mm, 1.3 mm, 1.4 mm, 1.5 mm, 1.6 mm, 1.7 mm, 1.8 mm 1.9 mm or 2.0 mm In some embodiments, the height 135 is about 1 mm, the height 136 is about 2 mm, and the height 401 is about 0.8 mm. Other suitable heights can also be used.

As illustrated in FIGS. 2-4, the height 136 of the support portions 141 of fingers 122 and 124 can be less than the length 133 of central tines 126a (for example, less than about 80%). This can permit the base portion 120 of the clip 102 to be positioned relatively close to the outer surface of the vessel wall 116 when the clip 102 is attached. In some embodiments, the support portions 141 can have different sizes or may be eliminated (e.g., with the tines 126a,b attaching directly to the base portion 120). In other embodiments, height 136 can be approximately equal to or greater than the length 133 of central tines 126a. The support portions 141 can include smoothly contoured sides 143, as illustrated, to diminish the likelihood that the support portions 141 will pierce the vessel wall 116 and/or cause trauma to the vessel wall 116. In the illustrated embodiment, the outer surface of the support portions 141 is curved (e.g., similar in curvature to the outer surface of the base portion 120). In some embodiments, the outer surface of the support portions 141 can be flat or can be shaped in a way different from the outer surface of the base portion 120.

FIG. 5 is a side-view of clip 102 in a closed configuration. In a deployed state, clip 102 can define an angle θ 130 between a central axial line or an edge in fingers 122, 124 and a peripheral surface or an edge 131 of base 120. Angle θ 130 can be selected to assist in determining the applied closure force and to facilitate removal of clip 102 in embodiments using temporary closure, as explained further below. Angle θ 130 also can be selected to assist in determining the overall depth of penetration by the tines 126a, 127b into the vessel wall 116. For example, a smaller angle will generally produce a more shallow penetration and a larger angle will generally produce a deeper penetration. In some embodiments, Angle θ 130 can be greater than or equal to about 300 and/or less than or equal to about 70°. In some embodiments, Angle θ 130 can be about 30°, 35°, 40°, 45°, 50°, 55°, 60°, 65°, or 70°. In a particular example, Angle θ 130 can be about 50°. Other appropriate angles can also be used. In some embodiments, as illustrated, the flexion regions 400 can bend while other structures remain substantially unchanging or intact.

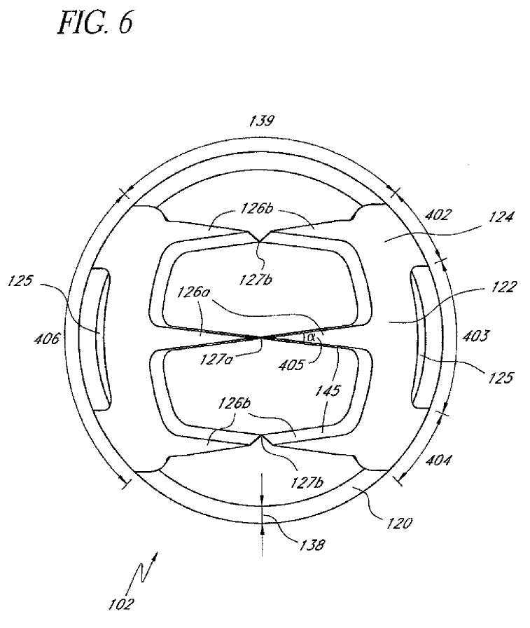

FIG. 6 is a bottom view of clip 102 in a closed configuration. As illustrated, opposed pairs of tines 126a, 126b can be configured to contact one another or to draw very close to each other (e.g., within a distance equivalent to about the thickness 137 of each tine 126a, 126b) in the closed configuration. In other embodiments, the tines 126a, 126 need not be configured to move very close to each other in the closed configuration. In some embodiments, base portion 120 has a side thickness 138 which can be greater than or equal to about 0.1 mm and/or less than or equal to about 0.5 mm. In some embodiments, base portion 120 has a side thickness 138 of about 0.1 mm, 0.2 mm, 0.3 mm, 0.4 mm or 0.5 mm. In some embodiments, base portion 120 has a side thickness 138 of about 0.2 mm. As illustrated, in some embodiments, all portions of the clip 102 can share approximately the same thickness. The thickness can also vary between different portions of the clip 102 in appropriate circumstances. For example, referring to FIG. 4, tines 126a-b can have a thickness 137 which can be less than thickness 138 of the base portion 120 to facilitate penetration of the vessel wall 116.

Base portion 120 can define an outer diameter and an inner diameter. For example, the outer diameter can be greater than or equal to about 3 mm and/or less than or equal to about 7 mm, and the inner diameter can be greater than or equal to about 2.5 mm and/or less than or equal to about 6.5 mm In some embodiments, the outer diameter is about 3.0 mm, 3.5 mm, 4.0 mm, 4.5 mm, 5.0 mm, 5.5 mm, 6.0 mm, 6.5 mm or 7.0 mm. In some embodiments, the inner diameter is about 2.5 mm, 3.0 mm, 3.5 mm, 4.0 mm, 4.5 mm, 5.0 mm, 5.5 mm, 6.0 mm or 6.5 mm. In some embodiments, the outer diameter is about 5.3 mm and the inner diameter is about 4.8 mm. Other suitable diameters can also be used. Different size clips can be used depending on the specific tissue compression or closure application for which they are being used and to account for different anatomical sizes, such as differences in the thickness or diameter of the vessel wall 116. In some instances, a plurality of different-sized clips 102 can be provided to health care professionals to allow for variability and increased precision in diminishing trauma and increasing the appropriate closure force for a particular patient. Moreover, a clip size also can be selected to accommodate the tubular medical device over which the clip will be advanced. In embodiments effecting arteriotomy closure, the clip's inner diameter should be large enough to be advanced over a standard commercial introducer.

As illustrated in FIG. 6, the tines can have straight edges 145 and define an inner angle α 405. Angle α 405 can be selected to help adjust an insertion force required to cause penetration of the tines 126a, 126b into or withdrawal of the tines 126a, 126b from the vessel wall 116. In some embodiments, angle α 405 can be greater than or equal to about 3° and/or less than or equal to about 15°. In some embodiments, angle α 405 can be about 3°, 4°, 5°, 6°, 7°, 8°, 9°, 10°, 11°, 12°, 13°, 14° or 15°. In some embodiments, angle α 405 can be about 9°. Other suitable angels can also be used. The widths of the tips 127a, 127b of tines 126a, 126b can also be adjusted to determine a required insertion force. In certain embodiments, the width of tips 127a, 127b can be greater than or equal to about 0.03 mm, and/or less than or equal to about 0.09 mm. In certain embodiments, the width of tips 127a, 127b can be about 0.03, 0.04, 0.05, 0.06, 0.07, 0.08 or 0.09 mm. in certain embodiments, the width of tips 127a, 127b can be about 0.06 mm. Other suitable tip widths can also be used. In certain embodiments, the edges of the tines 126a, 126b can be curved, segmented, or define different angles at different portions. In certain embodiments, the tines 126a, 126b can include barbs, protrusions, or other elements configured to resist withdrawal from the vessel wall 116. The barbs can be sized or configured to provide sufficient resistive force to prevent accidental removal of the clip 102 during partial deployment of the clip 102 as explained in more detail below. In certain embodiments, the resistive force provided by the barbs can also be sufficiently small to permit atraumatic removal of the clip 102.

For embodiments in which the base 120 is substantially circular, arc 406 corresponds to the circumferential width of fingers 122 and 124. In the illustrated embodiment, arc 406 subtends an approximately 90° angle. In some embodiments, arc 406 can subtend an angle greater than or equal to about 60° and/or less than or equal to about 90°. In some embodiments, arc 406 can subtend an angle of about 60°, 65°, 70°, 75°, 80°, 85°, or 90°. Other angles can also be used. Arc 403 corresponds to a circumferential width of window portions 125. In some embodiments, arc 403 can subtend an angle between greater than or equal to about 15° and less than or equal to about 30°. In some embodiments, arc 403 can subtend an angle of about 15°, 16°, 17°, 18°, 19°, 20°, 21°, 22°, 23°, 24°, 25°, 26°, 27°, 28°, 29° or 30°. In certain embodiments, arc 403 can be less than or equal to about one-half the length of arc 406. Connecting portions of fingers 122 and 124 adjacent to the window portions 125 can have widths defined by arcs 402 and 404. Arc 139 corresponds to the separation distance between fingers 122 and 124. In the illustrated embodiment, arc 139 subtends an angle of approximately 90°. In some embodiments, arc 139 can subtend an angle greater than or equal to about 60° and/or less than or equal to about 90°. In some embodiments, arc 139 can subtend an angle of about 60°, 65°, 70°, 75°, 80°, 85°, or 90°. Other angles can also be used. In some embodiments, as illustrated, the shape and/or orientation of the base portion are substantially or entirely unchanged in the transition between an open or pre-deployed state and a closed or deployed state.

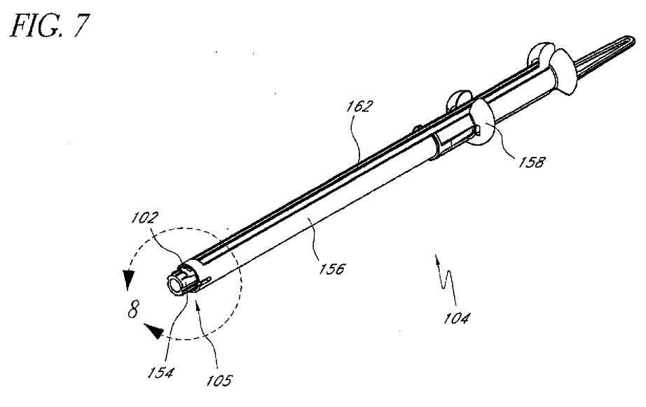

FIG. 7 is a perspective view of deployment instrument 104 with clip 102 in the open or pre-deployed position attached to a distal end thereof. The configuration illustrated in FIG. 7 is generally an initial or starting configuration before insertion of the deployment instrument 104 into a patient. The deployment instrument 104 with a pre-loaded clip 102 can be provided to the physician in a sterilized package in this general configuration. In certain embodiments, the deployment instrument 104 can be constructed with three basic components: inner tubular member 154, outer tubular member 156, and pressure element 158.
(b) Deployment Instrument

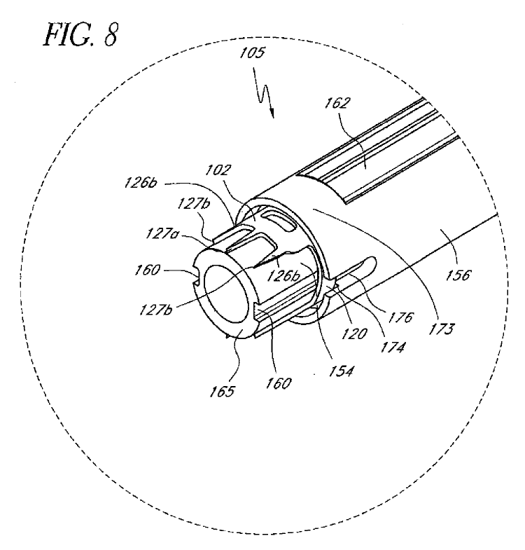

FIG. 8 provides a more detailed view of the distal end 105 of the deployment instrument 104, which is configured to receive clip 102 and generally maintain it in an open configuration until deployed. In the illustrated embodiment, the tines 126a, 126b are substantially parallel with a central axis of the inner tubular member 154, and the distal ends 127a, 127b of the tines 126a, 126b are substantially aligned with the distal end 165 of the inner tubular member 154. In other embodiments, the distal ends 127a, 127b of the tines 126a, 126b can extend slightly beyond the distal end 165 of the inner tubular member 154. Alternatively, clip 102 can be located more proximally while the deployment instrument 104 is in its initial configuration with the distal ends 127a, 127b of the tines 126a, 126b being proximally spaced from the distal end 165 of the inner tubular member 154. As will be described in more detail below, the inner diameter of the base 120 of the clip 102 can be positioned close to or in contact with the outer diameter of the distal end 165 of the inner tubular member 154, and the outer diameter of the base 120 of the clip 102 can be positioned close to or in contact with the inner diameter of the distal end 173 of the outer tubular member 156. A radially inwardly directed restoring force exerted by the tines 126a, 126b in the open configuration increases the friction between the inner surfaces of the clip 102 and the outer surface of the inner tubular member 154, generally preventing the clip 102 from readily sliding away from its position between the inner and outer tubular members 154, 156.

Distal end 173 of outer tubular member 156 can include an interior ledge or countersink 174 configured to receive and abut against the base 120 of clip 102. As will be explained in more detail below, when the assembled deployment instrument 104 is advanced to the tissue closure site and the inner tubular member 154 is axially withdrawn in the proximal direction from the outer tubular member 156, a distally directed reaction force is exerted by countersink 174 against the base 120 of the clip 102, preventing the clip 102 from also moving in the proximal direction. When the distal end 165 of the inner tubular member 154 is moved in the proximal direction past the base 120 of the clip 102, the contacting or adjacent relationship between the clip 102 and the inner and outer tubular members 154, 156 is interrupted and the clip 102 is released from the deployment instrument 104. In certain embodiments, the use of countersink 174 can permit the outer tubular member 156 to avoid contact with or otherwise to protect all or a portion of clip 102 during advancement prior to deployment. In other embodiments, countersink 174 can be omitted and the distal-most surface of outer tubular member 156 can be configured to contact base 120 to force off or otherwise permit removal of the clip 102 from the deployment instrument 104.

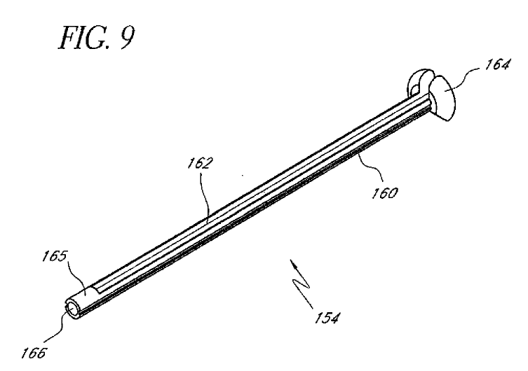

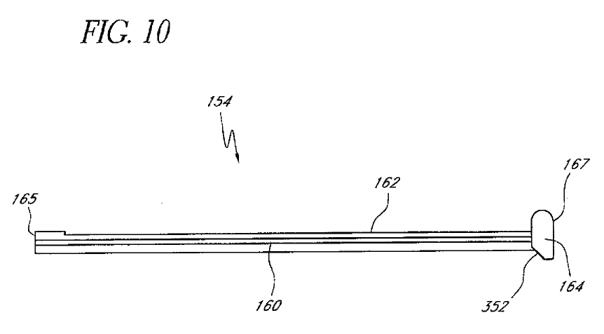

FIGS. 9-11 are illustrations of an example of the inner tubular member 154 separated from the outer tubular member 156 before the configuration illustrated in FIGS. 7-8 is assembled. Inner tubular member 154 defines an inner lumen 166 which is configured to receive a tubular medical device such as a vascular introducer 108. Elongate slot 162 allows at least a portion of the deployment instrument 104 to be tilted away from and axially separated from the proximal portion of the vascular introducer 108 by a medical professional without detaching the instrument 104 entirely from the tube section 110. See, e.g., FIG. 1. This configuration permits the medical professional to position the deployment instrument 104 out of the way while the desired interventional or diagnostic procedure is performed. In the illustrated embodiment, axial grooves 160 run along the length of the outer surface of inner tubular member 154 and are configured to mate with axial protrusions 168 (see FIG. 13) formed on an inner surface of outer tubular member 156. This mating configuration can prevent inner tubular member 154 from rotating relative to outer tubular member 156 and can help to align elongate slot 162 of inner tubular member 154 and elongate slot 170 of outer tubular member 156.

The proximal end of inner tubular member 154 can include a handle 164 which may be gripped by the medical professional, for example, to withdraw inner tubular member 154 during deployment. The handle is generally configured for handling by a user and for enabling a user to achieve motion or operation of a distal end in response to the user's control of the handle. As illustrated, handle 164 can be generally circular with a flattened lower end to facilitate delatching of the stop mechanism during complete deployment as explained below. Other shapes and configurations can also be used. The upper portion of handle 164 includes a cut-out portion 350 which is aligned with and merges with elongate slot 162. Lower portion of handle 164 includes a recess 169 to accommodate tab 172 of the outer tubular member 156. The distal end of handle 164 includes distal faces 354 which can be substantially flat. Faces 354 are configured to abut the proximal-most edge of the tube section of outer tubular member 156 to prevent over-insertion of inner tubular member 154 into outer tubular member 156. Proximal faces 167 of handle 164 can be substantially flat and are configured to abut stops 175 on tab 172 during partial deployment. Lower portion of the handle 164 can include angled surfaces 352.

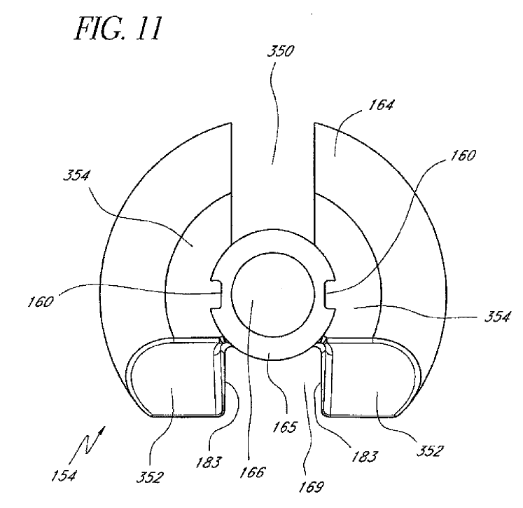

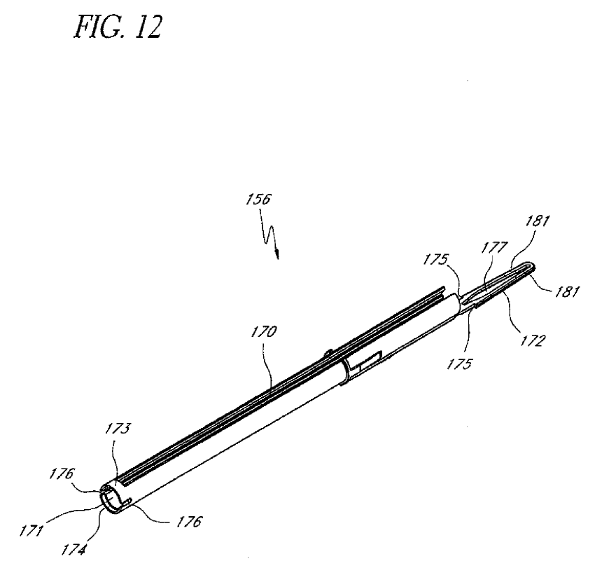

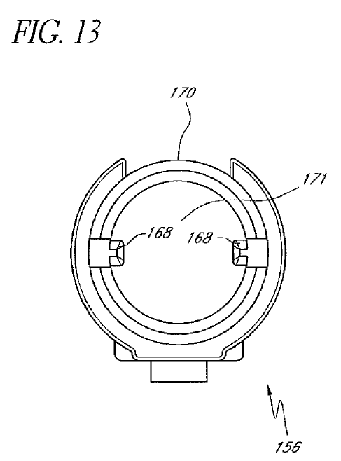

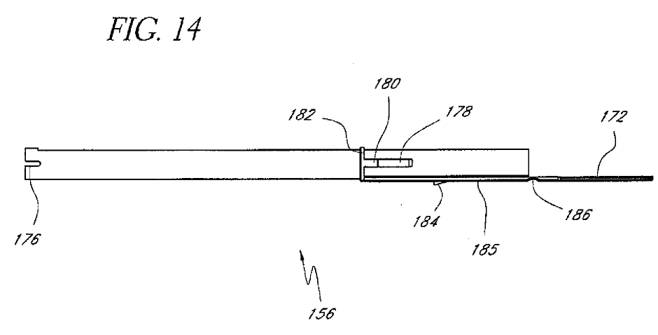

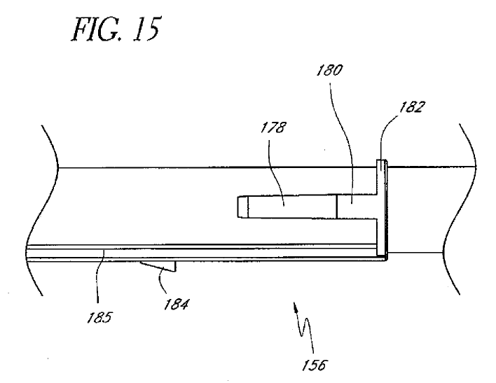

FIGS. 12-16 illustrate an example of an outer tubular member 156 separated from the inner tubular member 154 before the configuration illustrated in FIGS. 7-8 is assembled. Outer tubular member 156 defines an inner lumen 171 configured to receive inner tubular member 154. An elongate slot 170 runs along a length of outer tubular member 156 and provides access to the interior of inner lumen 171. Elongate slot 170 of outer tubular member 156 is configured to align with elongate slot 162 of inner tubular member 154. Distal end 173 of outer tubular member 156 can include one or more slots 176 to provide side access to clip 102 while deployment instrument 104 is in its initial configuration.

A securing or movement-limiting structure such as tab 172 extends from a proximal end of outer tubular member 156. Tab 172 includes stop surfaces 175 configured to abut the proximal faces 167 on handle 164 during partial deployment as explained in more detail below. Tab 172 can include two tapered arms 181 surrounding a window portion 177 to facilitate assembly of the deployment instrument 104 as explained further below. Tab 172 can also include a recessed, weakened, or hinge portion 186 to facilitate bending. In certain embodiments, tab 172 can be relatively rigid with the exception of weakened portion 186. In certain embodiments, bending of tab 172 can be configured to occur substantially at weakened portion 186. In certain embodiments, tab 172 can be relatively long. For example, tab 172 can be at least about 20 mm. A long tab 172 can facilitate handling by the medical professional. A long tab 172 can also increase the leverage applied by the medical professional to effectuate bending.

The deployment instrument can include a pressure sensitive structure which can comprise, in one example, pressure tapers 178 formed on an outer surface of outer tubular member 156 and flexible tabs 188 of pressure element 158. Outer tubular member 156 can also include a pressure sensitive structure such as an axial protrusion 185 extending from a proximally-located outer surface. As illustrated, axial protrusion 185 can be located in a substantially diametrically opposite position from elongate slot 170, although other configurations are possible. A ramp or one-way tapered lock 184 extends from axial protrusion 185. A stop, 182 which can be generally annular in shape, extends from an outer surface of outer tubular member 156. The outer surface of outer tubular member 156 also includes pressure tapers 178. Pressure tapers 178 can terminate in substantially flat surfaces 180. Surfaces 180 can be adjacent to and in contact with annular stop 182. As illustrated in FIG. 16, outer tubular member 156 can include two pressure tapers 178 located in a substantially diametrically opposite position from one another on the generally circular outer tubular member 156. Also as illustrated, pressure tapers 178 can be positioned at approximately equal circumferential distances from elongate slot 170 and axial protrusion 185. Other configurations are possible.

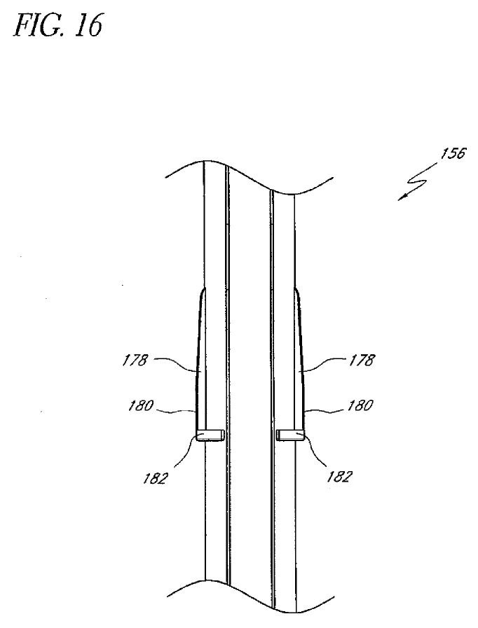

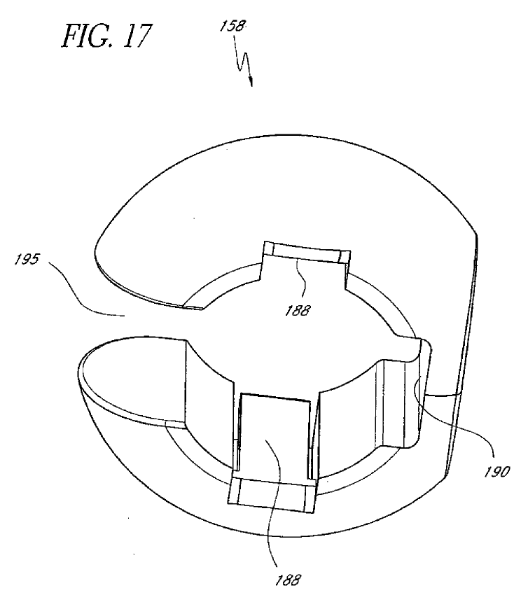

FIG. 17 provides a detailed illustration of a pressure element 158, which in some embodiments can be a generally ring-shaped element configured to be received on an outer surface of outer tubular member 156. In certain embodiments, as illustrated, pressure element 158 can be a separate element from outer tubular member 156. In other embodiments, pressure element 158 can be integrally formed with outer tubular member 156. As described in more detail below, pressure element 158 can be used to confirm that the medical professional is applying generally sufficient but not excessive pressure to safely begin deployment of the clip 102. Pressure element 158 can include a cut-out portion 105 aligned with elongate slots 162, 170 of the inner and outer tubular members 156, 154. Recess 190 can be configured to mate with axial protrusion 185 of outer tubular member 156 to keep the pressure element 158 properly aligned. An inner surface of pressure element 158 includes one or more flexible tabs 188. Flexible tabs 188 are configured to align with, and be advanced over, pressure tapers 178 of outer tubular member 156.

During assembly of deployment instrument 104, pressure element 158 can be advanced over the proximal end of outer tubular member 156 and over one-way tapered lock 184. Recessed portion 190 and/or lock 184 can be configured to flex or temporarily deform sufficiently to accommodate this procedure. Alternatively, lock 184 or other locking means can be formed on, or secured to, outer tubular member 156 after positioning of pressure element 158. Tapered lock 184 prevents pressure element 158 from moving too far in a proximal direction with respect to outer tubular member 156. Inner tubular member 154 can then be inserted into the inner lumen 171 of outer tubular member 156 from the outer tubular member's proximal end. As the inner tubular member 154 is inserted into outer tubular member 156, inner surfaces 183 (see FIG. 11) of the lower portion of handle 164 adjacent to recess 169 begin to come into contact with tapered arms 181 of tab 172. The continued advancement of inner tubular member 154 distally causes surfaces 183 to apply an inwardly-directed force to arms 181. Window 177 permits arms 181 to resiliency flex inwardly until handle 164 has been advanced distally of stops 175. Inner tubular member 154 can then be advanced further until distal faces of handle 354 contact the proximal-most edge of the tube section of outer tubular member 156.
(c) Methods of Use

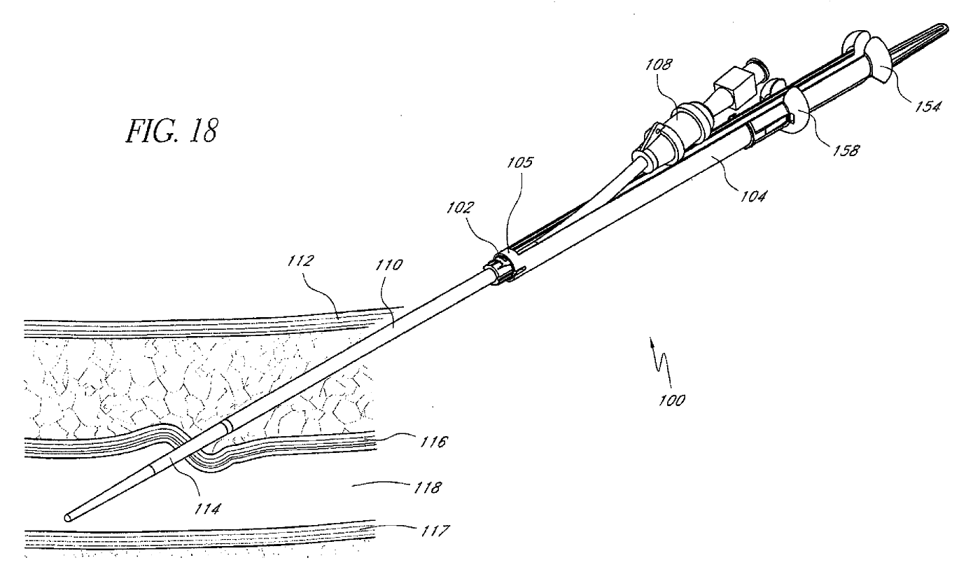

An example of a method for using deployment instrument 104 and clip 102 will now be described. FIG. 18 illustrates a deployment instrument 104 in an initial configuration loaded onto a vascular introducer 108 that has been inserted into a patients blood vessel 118. The deployment instrument 104 can also be configured for use with other medical devices such as, for example, tubular or elongate dilators, trocars, endoscopes, catheters, guide wires, needles, tubes, sheaths, combination or other. The tubular medical device 108 is first inserted through the inner diameter of the deployment instrument 104 which has been loaded with clip 102. The tubular medical device 108 can then be inserted through the skin and into the desired vessel 118 using any of a number of known methods, such as, for example, the Seldinger method. The desired interventional or diagnostic procedure is then performed. The deployment instrument 104 can be temporarily moved to the side as illustrated so as not to interfere with the medical procedure. For example, the deployment instrument 104 can be moved toward the back or proximal end of the introducer sheath 108 as shown in FIG. 18. Slots 162 and 170 (see FIGS. 7 and 12) facilitate this positioning.

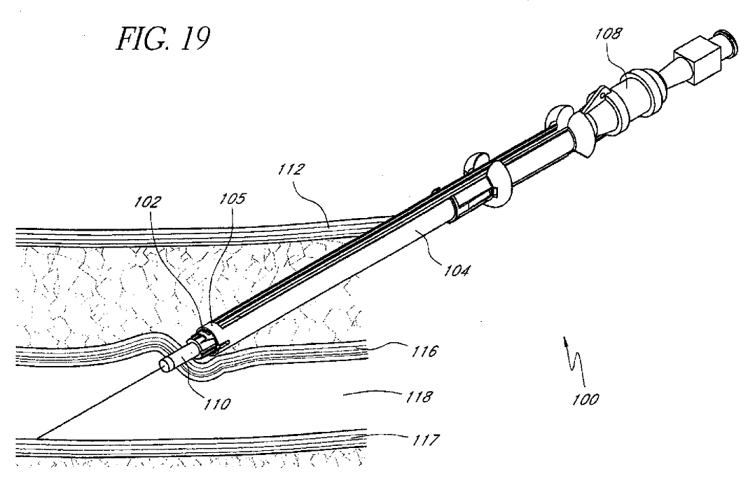

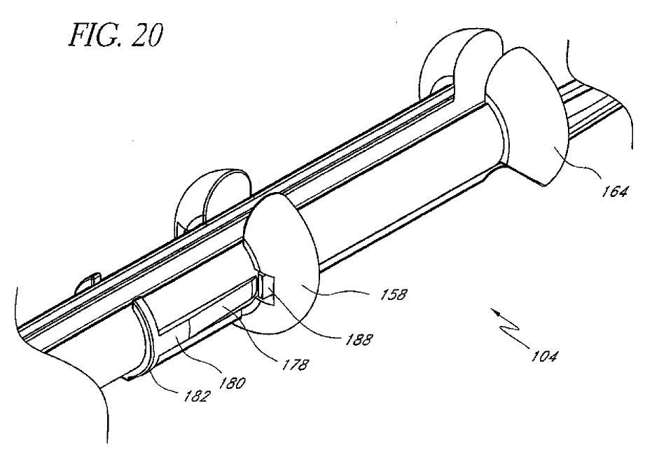

With reference to FIGS. 19-20, deployment instrument 104 is advanced forward along the introducer sheath through the percutaneous opening 112 until the distal end 105 of the deployment instrument 104 contacts the vessel wall 116. At this state along the pressure sensitive structure on the outside of outer tubular member 156, pressure element 158 is in its initial, non-advanced configuration as shown in FIG. 20. In certain embodiments, a dilator that was previously removed or a new dilator or other elongate member can be inserted into the inner lumen of the vascular introducer 108 to provide mechanical support and resistance to kinking of the introducer 108. Reinsertion of the dilator may thus facilitate the advancement of deployment instrument 14 over the introducer 108.

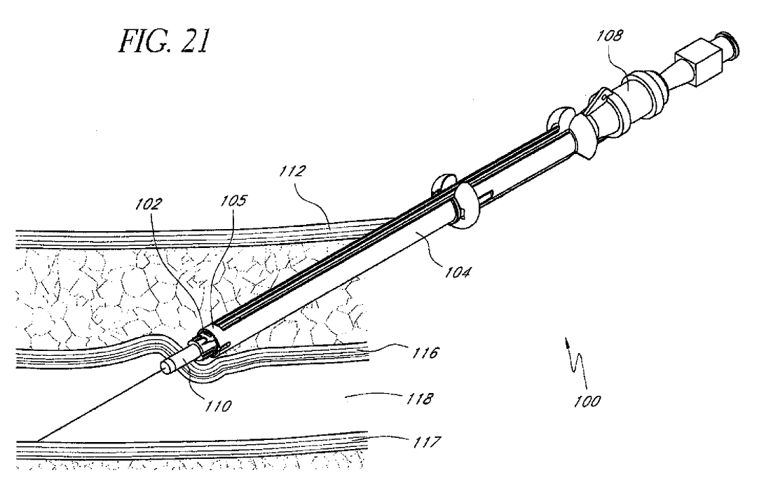

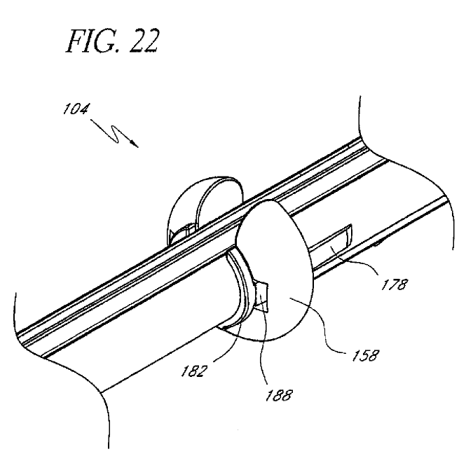

With reference to FIGS. 21-22, pressure element 158 is then manually advanced distally until it reaches stop 182, indicating to the medical professional that appropriate force is being applied between the deployment instrument 104 and the vessel wall 116 to begin deployment. FIG. 22 is a close up view of the pressure element 158 in its fully advanced configuration. As the pressure element 158 is advanced distally, flexible tabs 188 are subjected to greater flexion as they advance up pressure tapers 178. Thus, advancing the pressure element 158 can require an increasing amount of applied force. Pressure tapers 178 generally flare outward until reaching flat surfaces 180. Stop 182 generally prevents pressure element 158 from advancing distally beyond this point. The amount of applied force required to fully advance the pressure element 158 can be adjusted by altering one or more of the number, size, width and rigidity of tabs 188, the angle of incline of pressure tapers 178 and the height of surfaces 180. In certain embodiments, the deployment instrument 104 can require at least about 10 ounces of force to safely begin deployment of the clip 102. Thus, in certain embodiments, pressure element 158 can require at least about 10 ounces of force to be fully advanced. In other embodiments, the deployment instrument 104 can require between about 3 ounces of force and about 64 ounces of force to safely begin deployment of the clip 102. In some embodiments, less than about 3 ounces of force is required. In other embodiments, the deployment instrument 104 can require about 3 ounces of force, about 4 ounces of force, 5 ounces of force, about 6 ounces of force, about 7 ounces of force, about 8 ounces of force, about 9 ounces of force, about 10 ounces of force, about 11 ounces of force, about 12 ounces of force, about 13 ounces of force, about 14 ounces of force, about 15 ounces of force, about 16 ounces of force, about 17 ounces of force, about 18 ounces of force, about 19 ounces of force, about 20 ounces of force, about 21 ounces of force, about 22 ounces of force, about 23 ounces of force, about 24 ounces of force, about 25 ounces of force, about 26 ounces of force, about 27 ounces of force, about 28 ounces of force, about 29 ounces of force, about 30 ounces of force, about 31 ounces of force, about 32 ounces of force, about 33 ounces of force, about 34 ounces of force, about 35 ounces of force, about 36 ounces of force, about 37 ounces of force, about 38 ounces of force, about 39 ounces of force, about 40 ounces of force, about 41 ounces of force, about 42 ounces of force, about 43 ounces of force, about 44 ounces of force, about 45 ounces of force, about 46 ounces of force, about 47 ounces of force, about 48 ounces of force, about 49 ounces of force, about 50 ounces of force, about 51 ounces of force, about 52 ounces of force, about 53 ounces of force, about 54 ounces of force, about 55 ounces of force, about 56 ounces of force, about 57 ounces of force, about 58 ounces of force, about 59 ounces of force, about 60 ounces of force, about 61 ounces of force, about 62 ounces of force, about 63 ounces of force, or about 64 ounces of force to safely begin deployment of the clip 102. In certain embodiments, the deployment instrument can be configured to make an audible “click” or otherwise produce an audio, visual, or tactile signal when the pressure element 158 has fully advanced.

In some embodiments, other pressure-sensitive structures such as a pressure or force gauge can be used to verify that adequate pressure is applied. The deployment instrument can use a spring in place of, or in addition to, a taper element. A first end of the spring can be secured to a slidable element. A second end can be attached to a distal point on the outer tubular member. The slidable element can be used to compress the spring, thus applying force to the outer tubular member. A combination or other means to confirm sufficient contact and pressure between the deployment instrument and vessel can also be included. In certain embodiments, the deployment instrument can include a grasping tool configured to assist in securing the distal end of the deployment instrument to the vessel. In certain embodiments, the medical professional can observe a backflow of blood through a channel or window in the deployment instrument following removal of the tubular medical device to confirm proper placement on the vessel. Blood can be configured to flow through the central channel of the deployment instrument. In certain embodiments, a clear channel can be provided to receive blood flow. One or more sensors can be provided to verify proper placement and/or pressure.

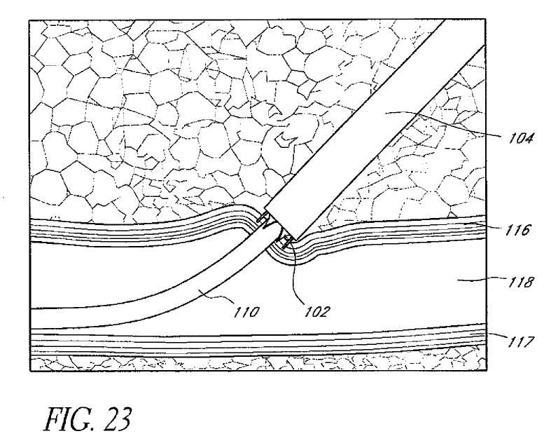

FIG. 23 shows the deployment instrument 104 with clip 102 in a partially-deployed configuration. In a partially-deployed state, tines 126a, 126b can pierce the vessel wall 116 and the clip 102 remains attached to the deployment instrument 104 in a substantially open configuration. The medical professional partially deploys the clip 102 by beginning to withdraw inner tubular member 154. The medical professional can maintain adequate pressure on pressure element 158 (e.g. pressure sufficient to maintain pressure element 158 in its fully advanced configuration) while withdrawing inner tubular member 154. Handle 164 can be used to withdraw the inner tubular member 154. For example, the medical professional can apply distally-directed pressure to the pressure element 158 with one hand while partially withdrawing handle 164 with the remaining hand. The ledge or countersink 174 on outer tubular member 156 prevents clip 102 from being withdrawn along with the inner tubular member 154. Thus, as the inner tubular member 154 is withdrawn, the tines 126a,b begin to extend beyond the distal end 165 of inner tubular member 154. The continued application of pressure on pressure element 158 (and thus outer tubular member 156) generally forces the tines 126a,b to pierce the vessel wall 116. In certain embodiments, the pressure element 158 can include a means to prevent inner tubular member 154 from being withdrawn unless and until pressure element 158 is fully advanced.

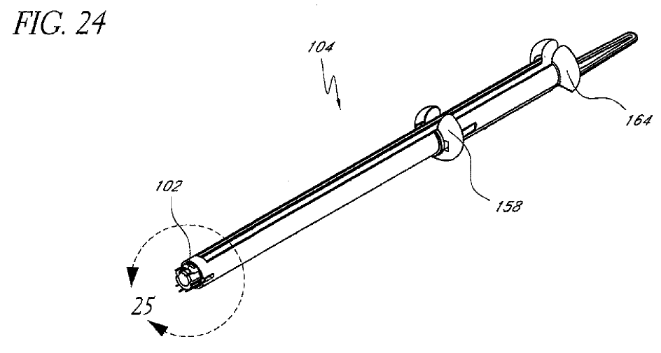

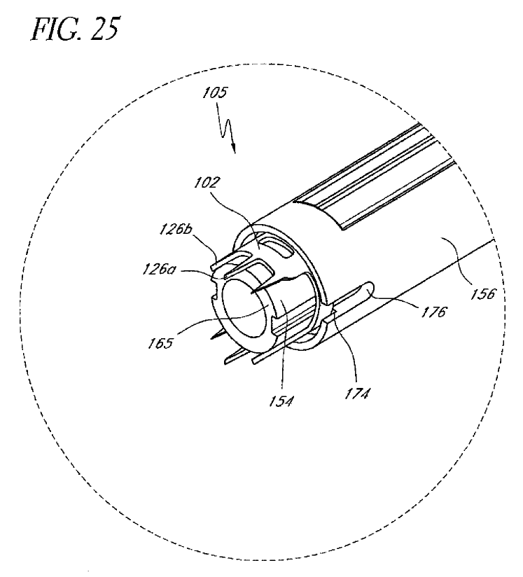

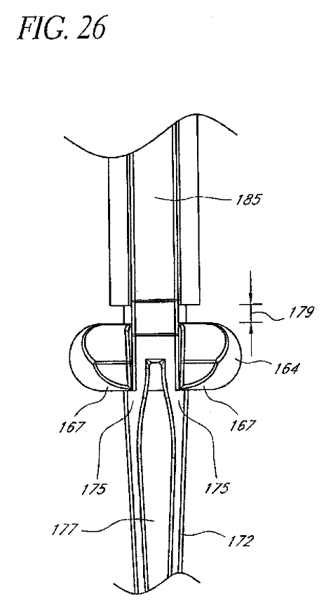

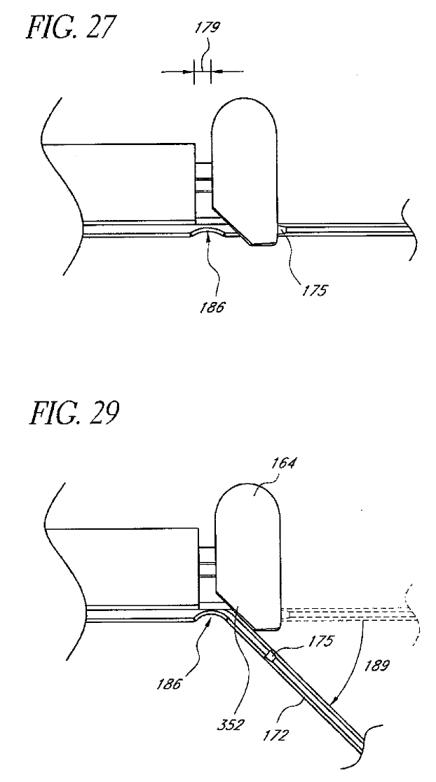

FIGS. 24-29 illustrate an example of a method of producing partial deployment. FIG. 24 shows a perspective view of the deployment instrument 104 in a partially-deployed state, and FIG. 25 shows a close-up view of the distal end 105 of the deployment instrument 104 in its partially-deployed state. Handle 164 can be withdrawn until the proximal face 167 of handle 164 contacts the stops 175, generally arresting further withdrawal as shown in FIGS. 26 and 27. Stops 175 generally prevent the medical professional from fully deploying the clip prematurely and ensure the clip 102 is partially deployed to an appropriate depth. Stop 175 is configured to allow the handle 164 to travel a known, limited distance 179. In embodiments where the tips 127a,b of tines 126a,b are initially aligned with distal end 165 of inner tubular member 154, distance 179 can correspond to a depth of the tines' insertion into the vessel wall 116. In certain embodiments, distance 179 can be greater than or equal to about 0.5 mm and/or less than or equal to about 4 mm. In certain embodiments, distance 179 can be about 0.5 mm, 0.6 mm, 0.7 mm, 0.8 mm, 0.9 mm, 1.0 mm, 1.1 mm, 1.2 mm, 1.3 mm, 1.4 mm, 1.5 mm, 1.6 mm, 1.7 mm, 1.8 mm, 1.9 mm, 2.0 mm, 2.1 mm, 2.2 mm, 2.3 mm, 2.4 mm, 2.5 mm, 2.6 mm, 2.7 mm, 2.8 mm, 2.9 mm, 3.0 mm, 3.1 mm, 3.2 mm, 3.3 mm, 3.4 mm, 3.5 mm, 3.6 mm, 3.7 mm, 3.8 mm, 3.9 mm, or 4.0 mm. In certain embodiments, distance 179 can be about 2 mm. Other suitable distances can also be used. Distance 179 can be different depending on the specific application or clip being used.

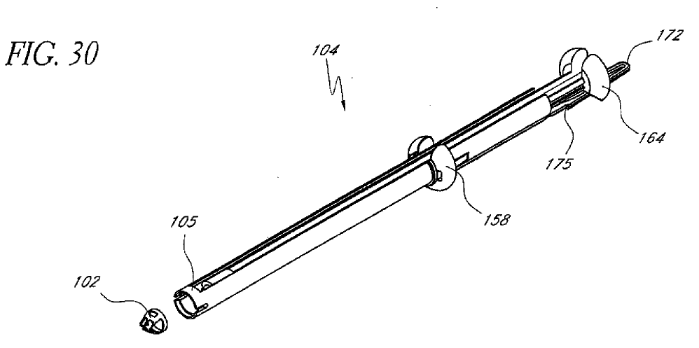

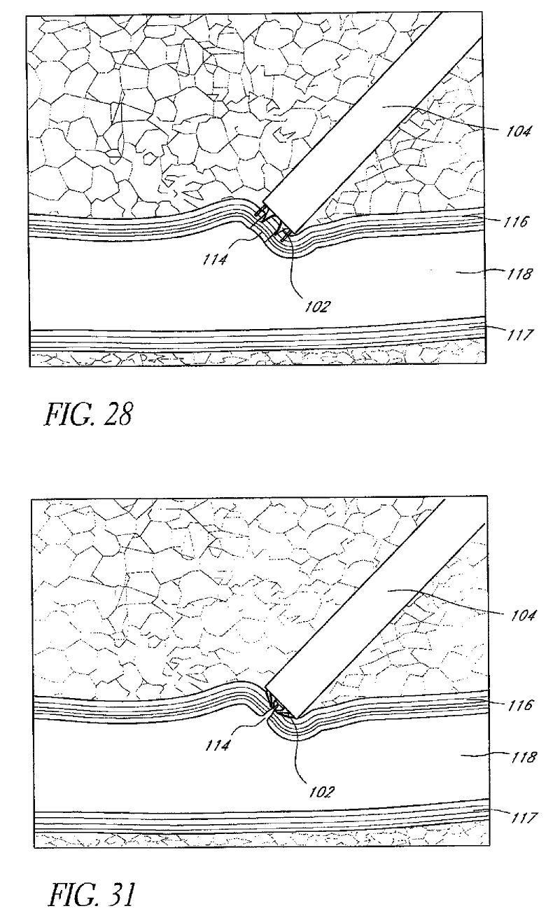

With the clip 102 partially deployed in the vessel wall 116, the tubular medical device 108 is no longer needed to guide the deployment instrument 104 to the arteriotomy and hence the tubular medical device 108 can then be removed from the vessel 118 as shown in FIG. 28. Removing the tubular medical device 108 prior to full deployment prevents the clip 102 from closing over the tubular medical device 108. Partially deploying the clip 102 helps to position the deployment instrument 104 more accurately and temporarily secure it in place while the tubular medical device 108 is removed.

Once the tubular medical device 108 is removed from the vessel, the stops 175 can be overcome by bending tab 172 in the direction of the arrow 189 shown in FIG. 29 to allow full linear movement of the inner tubular member 154. Tab 172 can thus operate as a releasing element, permitting the stops 175 to be overcome. Recessed or weakened portion 186 of tab 172 may facilitate bending. The flattened bottom portion and angled faces 352 of handle 164 can reduce the amount that tab 172 is required to bend in order to overcome stops 175. In some embodiments where deployment instrument 104 is disposable and configured for one-time use, tab 175 may be configured to snap off. Other appropriate stop means and methods for overcoming the stop means can be used.

With reference to FIGS. 30-31, the medical professional then continues to withdraw inner tubular member 154 until the clip 102 is forced off of or advanced past the distal end 105 of the deployment instrument 104. The opposed fingers 122, 124 of the clip 102 fold inwardly, drawing together sides of the vessel tissue from an outside surface of the vessel to close the arteriotomy 114 as shown in FIG. 31. Closing the arteriotomy can, but does not necessarily, result in complete mechanical closure of the opening. Instead, the term “close” in this context can refer to any facilitation of hemostasis. Thus, in certain embodiments, sides of the vessel tissue may not necessarily touch. Generally, the sides of the vessel tissue are brought closer together to reduce the size of the opening 114 in the vessel 118 and thereby facilitate hemostasis.

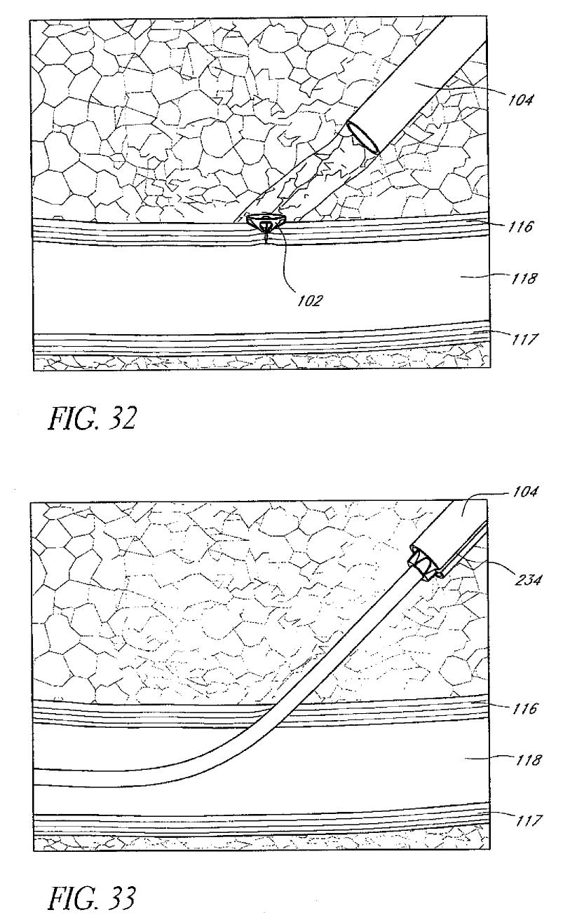

FIG. 32 shows the deployment instrument 104 being withdrawn following successful deployment of the clip 102. Clip 102 can be biocompatible and configured for permanent implantation. Accordingly, in certain embodiments a patient may be discharged following confirmation of successful clip deployment and hemostasis.

In some embodiments, vascular closure system 100 can be completely or substantially extravascular in that the deployment instrument or closure device is not required to penetrate into the interior region of blood vessel 118. This can reduce or eliminate the amount of foreign material introduced into contact with the patient's blood stream, thus reducing the risk of infection, blockage, or other complications. For example, in certain embodiments a posterior support is not required during deployment of the clip. In some systems, the use of posterior support may disadvantageously require that a portion of the deployment tool or closure device be positioned in the blood vessel during or following deployment. The use of a posterior support element within the vessel may require complicated mechanisms to facilitate its removal following deployment. The safe deployment of the clip without requiring posterior support can be facilitated through use of a partial deployment technique as described above and by the application of a controlled amount of external pressure via a pressure element or other pressure sensing means. In addition, the use of a clip with appropriately-sized tines to prevent overinsertion can also facilitate deployment without posterior support.

The system 100 described above can also be compatible with standard commercially available introducers already used in standard vascular interventional or diagnostic procedures. This can eliminate the need to purchase and use specialized and costly additional or different equipment or to change the way that the interventional or diagnostic procedures are performed, thus reducing the accompanying risks.

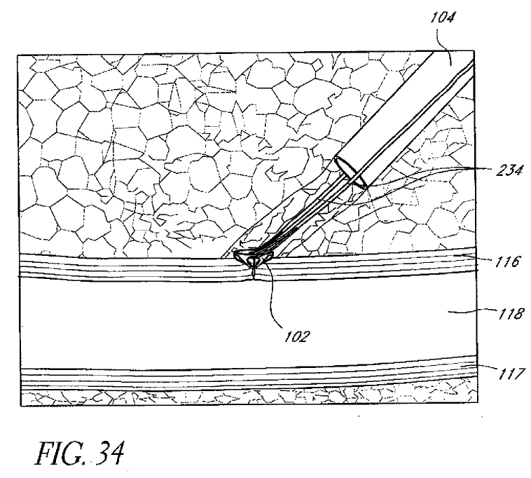

FIGS. 33-35 illustrate an example of a method of temporarily implanting a clip 102. In certain embodiments, a vessel closure clip 102 can be removable and configured for temporary implantation as illustrated in FIG. 33. In embodiments using temporary closure, one or more suture lines 234 or other suitable tethering means can be secured to the clip 102 and positioned along the outer surface of the outer tubular member 156 prior to insertion. The suture lines 234 can be tied to the clip 102 or looped through window portions 125 or other openings provided on the clip 102 for this purpose or attached in some other way. In certain embodiments, the clip 102 and deployment instrument 104 may be provided to the medical professional with suture lines 234 attached. In other embodiments, suture lines 234 may be attached by the medical professional prior to use. Slots 176 on the distal end 173 of outer tubular member 156 (see FIG. 8) can facilitate access to the clip 102 for the purposes of securing the suture lines 234 to the clip 102 after it is loaded onto the deployment instrument 104. The distal ends of axial grooves 160 on inner tubular member 154 can allow the suture lines to be passed under base portion 120. In certain embodiments, the suture lines 234 can be tied or secured to the clip before it is loaded on the deployment instrument 104. The suture lines 234 can run along the outer surface of outer tubular member 156 as shown in FIG. 33. In other embodiments, the suture lines 234 can run along an interior of the deployment instrument 104. In certain embodiments, the deployment instrument 104 can include channels specifically adapted to accommodate suture lines 234.

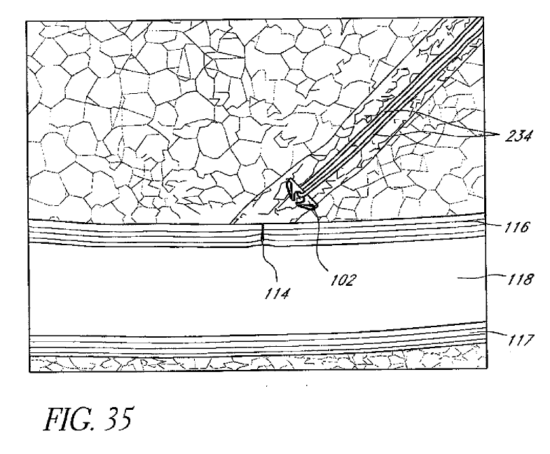

The removable clip 102 can be temporarily implanted using the procedure outlined above. The proximal ends of the suture lines 234 can be left extending outside of the patient's body while the clip 102 remains implanted. After a period of time sufficient to achieve hemostasis, the medical professional can pull on the suture lines 234 to remove the clip as seen in FIG. 35. The closure force of the clip can be configured so that force applied to the suture lines 234 causes the fingers 122, 124 to temporarily open, allowing the clip 102 to be safely removed without reopening the arteriotomy 114 or damaging the vessel wall 116. In certain embodiments, the clip 102 can include another or alternative release mechanism that can be triggered via the suture lines 234. The release mechanism can cause the fingers 122, 124 to open to facilitate removal of the clip 102. In embodiments using a shape memory clip, the clip can be cooled until it transforms to its martensite phase, making it more easily deformed and lowering the amount of force required to open the clip's fingers and withdraw it. The clip 102 can be cooled via insertion of a cold probe or via application of an externally-applied cold source such as an ice pack. In addition or in the alternative, an infusing syringe can be used to deliver a cooled liquid such as chilled saline to the clip. In certain embodiments, the clip 102 can exhibit a two-way shape memory effect and cooling the clip 102 can return it to its second memorized configuration which can be, for example, an open configuration. The clip's composition and treatment can be selected to achieve desired phase transition temperatures to facilitate such an approach.

The time required to achieve hemostasis can vary from patient to patient depending on a variety of factors including the patient's age, sex, medical condition, medications, and the presence of anti-clotting agents that can have been used during the medical procedure. Under certain conditions, clip 102 can be removed after about 10 minutes, after about 15 minutes, after about 20 minutes, after about 25 minutes, after about 30 minutes, after about 35 minutes, after about 40 minutes, after about 45 minutes, after about 50 minutes, after about 55 minutes, or after about 60 minutes. In some embodiments, clip 102 can be removed after about 1 h or more. Other suitable times can also be used.

In some embodiments, it can be desirable to use suture lines 234 even in clips intended for permanent implantation in order to enable emergency removal. In this arrangement, the medical professional can deploy the clip using the procedure described above. Once it is determined that the clip has been successfully deployed, the medical professional can cut the suture lines 234 and completely withdraw them from around the clip.
(d) Manufacture

The deployment instrument 104 can be partially or completely made from one or more of the following materials: polymers, including Nylon, polyamide, polycarbonate (e.g., Makrolon®), acrylonitrile butadiene styrene (ABS), polyester, polyethleneteraphthalate (PET), polyetherethereketone (PEEK™), polyimide, superelastic/shape memory polymers and metals, including spring steel, stainless steel, shape memory metal alloys including nickel titanium alloys (Nitinol), 17-7 PH, cobalt-chromium-nickel alloy (Elgiloy®), and nickel based alloys with chromium and iron (Inconel®). Other suitable materials can be used. The deployment instrument 104 can be completely or partially fabricated using one or more of the following methods: casting, extrusion, laminating, machining, molding (injection or other), sintering, or stereo lithography. Other suitable methods can be used.

As illustrated, in certain embodiments, the deployment instrument 104 can be constructed using relatively few components, e.g., an inner tubular member, an outer tubular member, and a pressure element. Each of the components can be produced inexpensively via injection molding. In certain embodiments, the deployment instrument 104 can be disposable and designed for single use. Alternatively, the deployment instrument 104 can be designed for repeated use following sterilization.

In certain embodiments the advancement/deployment tool can contain more than one clip, with the ability to deploy one or more clips at a time, and can include an indexing or other means to controllably deploy only one (or more) clips at a time. A multiple-clip embodiment can include at least two or more of the clips tethered together with a suitable tether. The tether can be elastic and/or able to be tensioned or otherwise configured to permit tissue between the two or more deployed clips to be pursed as the deployed clips are pulled (or drawn) towards one another. The tether can be permanently or temporarily tightened and secured at, for example, one or more ends of the tether to maintain the tension.

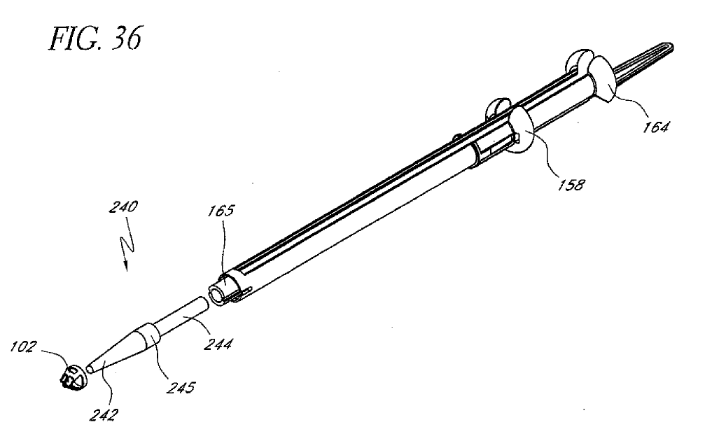

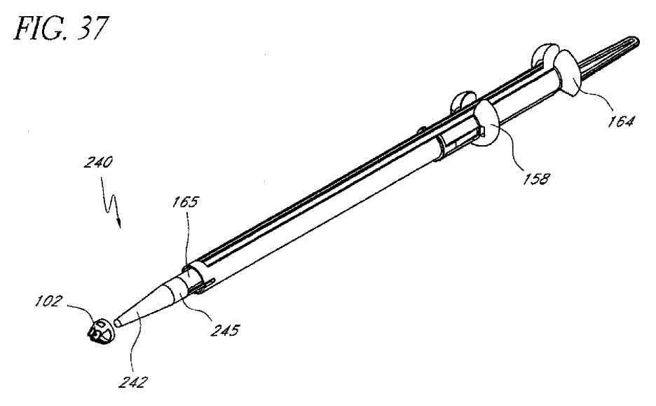

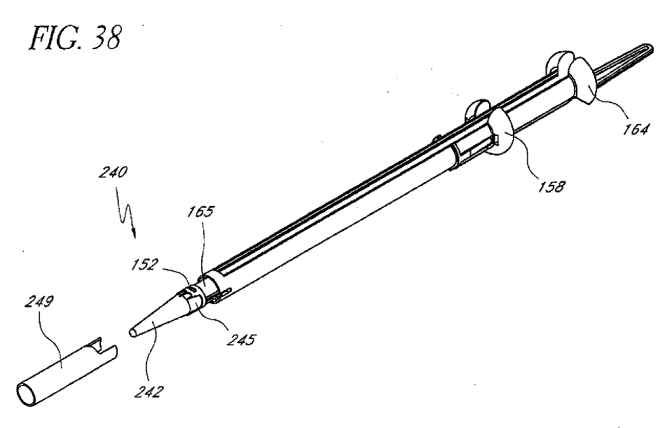

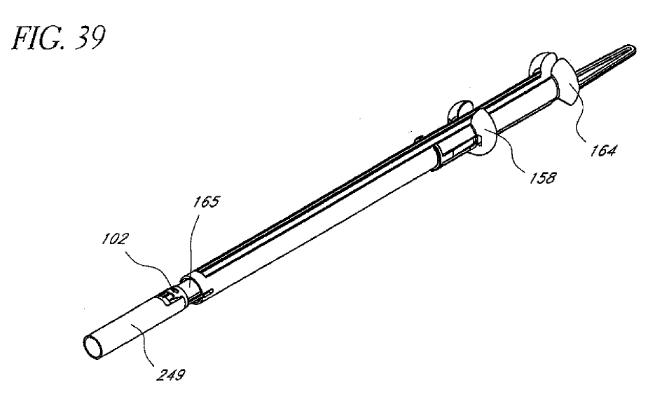

A method for loading the clip 102 onto the deployment instrument 104 will now be described with reference to FIGS. 36-39. A loading mechanism 240 can be used to facilitate loading the clip 102 onto the distal end 165 of inner tubular member 154. Loading mechanism 240 includes a proximal section 244 which mates with the inner tubular member's inner lumen as seen in FIG. 37. Clip 102 is then advanced over tapered distal section 242 of loading mechanism 240. Distal section 242 gradually forces apart the clip's fingers 122, 124 as shown in FIG. 38. The loading mechanism 240 can also include an intermediate section 245 with a substantially constant circumference which can be substantially equal to that of inner tubular member 154. A pusher mechanism 249 can be used to advance the clip over the loading mechanism 240 and onto the deployment instrument 104. Pusher mechanism 249 can include an end geometry configured to mate with the distal end of clip 102 as seen in FIG. 39. Once the clip 102 has been fully loaded onto the deployment instrument 104, the pusher mechanism 249 and loading mechanism 240 can be removed. In embodiments using a superelastic or shape memory clip, the clip 102 can be cooled until it undergoes a martensite phase transformation in order to facilitate the clip's deformation. During its martensite phase, the clip 102 is more easily deformed and thus the fingers 122, 124 can be more readily spread apart in order to load the clip 102 onto the deployment instrument 104. Such an approach can be used as an alternative to or in addition to the loading procedure described above.
(e) Guided Tissue Cutter

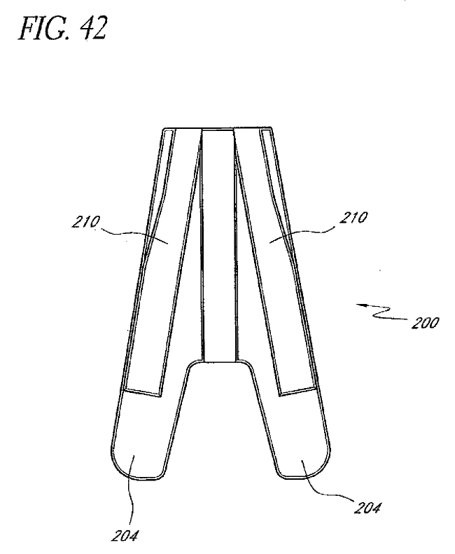

FIGS. 40-42 illustrate an example of a tissue opening widener such as a guided slidable tissue cutter 106, which can be used in a vessel closure system 100 in certain embodiments. After completing the desired medical procedure, the medical professional can temporarily attach tissue cutter 106 by clipping it onto the tube section 110 of the vascular introducer 108 as shown in FIG. 1. The tissue cutter 106 can then be slidably advanced along the vascular introducer sheath 108. The cutter 106 can be configured to make an incision of a precise depth and width at the site of the percutaneous opening 112 using sharp distal edges 203 of blades 202. The cutter 106 generally positions the edges 203 of the blades 202 at a specific orientation and distance from the tube 110 to permit a consistently and modestly sized entry point for the deployment instrument 104. A ledge such as mechanical stops 208 can ensure that the incision is not any deeper than needed to facilitate entry of the deployment instrument 104. Using the existing introducer sheath 108 as a guide for the slidable tissue cutter 106 also assists in ensuring proper placement of the incision. After making the incision, the slidable tissue cutter 106 can be removed from the side of the vascular introducer.

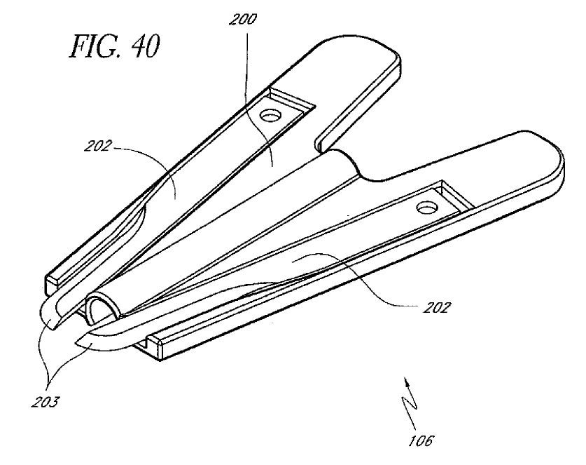

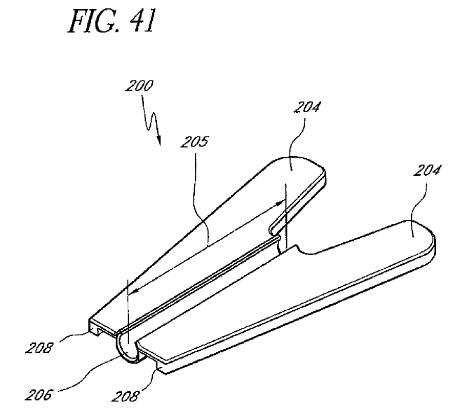

FIGS. 41-43 illustrate an example of a frame portion 200 which can form a component of a slidable tissue cutter 106. In certain embodiments, scalpel blades 202 can be secured to frame portion 200. In other embodiments, the cutter 106 can use specialized blades and/or be formed from a single piece. As illustrated, slidable cutter 106 includes two blades 202 positioned on lateral sides, such as in a diametrically opposite position from one another. In other embodiments, a single blade or three or more blades can be used. In certain embodiments, the cutting surfaces of each blade 202 can be static and configured to cut tissue without requiring interaction with a second cutting surface. In other embodiments, dynamic blades can be used.

Slidable cutter 106 can include a channel 206 with a partial circumferential cross-sectional geometry as shown in FIG. 43. This geometry enables a “snap-on” feature permitting the cutter 106 to be easily and temporarily attached to the tubular medical device and facilitating removal of the cutter 106 once the desired tissue has been cut. In other embodiments, a slidable cutter can use two mating pieces that clamp or snap together to facilitate temporary attachment and removal. In a preferred embodiment, channel 206 is sized so as to be compatible with any commercialized introducer sheath. The ends 208 of the frame portion 200 act as mechanical stops to control the depth of the incision. In some embodiments, handle portions 204 can extend beyond the end of channel 206 to facilitate handling by the medical professional at a distance from the sharp edges 203. Advantageously, such a configuration can facilitate the medical professional's control of the instrument without requiring an increase in the length 205 of channel 206. Most commercially available vascular introducers are between 11 and 13 cm long. Once inserted into a patient's vessel, the exposed portion of the introducer's tube section can be relatively small. Thus, it can be desirable to limit the amount of tube section that is taken up by the attached cutter and hence to reduce the length 205 of channel 206. The proximal ends of the handle portions 204 can be flared outwardly as illustrated to provide increased space between the cutter 106 and the tube 110 for improved manual access and manipulation, and to permit the deployment instrument 104 to be positioned as close axially as possible to the generally short exposed length of tube 110. The lateral edges of the cutter 106 can be tapered as illustrated.

Frame 200 can include recesses 210 sized to receive scalpel blades 202. The recesses 210 can be used to shield portions of the blades 202 not intended to out tissue. Scalpel blades 202 can be secured to frame 200 via one or more of a variety of known methods such as, for example, friction-fitting, mechanical interference fitting, sonic welding, adhesives, screws, clamps, and the like. As illustrated, scalpel blades 202 are configured to angle inward toward one another slightly. Such a configuration can help to ensure that the blades 202 cut tissue immediately adjacent to the percutaneous opening 112. In other embodiments, scalpel blades 202 can be oriented in a substantially parallel configuration. In some embodiments, the blades 202 can be adjustable, allowing a medical professional to adjust one or more of the incision's depth, width, and angle, and/or a collection of cutters 106 of different sizes can be provided for different applications. In certain embodiments, slidable tissue cutter 106 is configured to cut substantially only the patients skin. Fatty tissue located beneath the skin will generally move out of the way of the deployment instrument 104 with minimal resistance. Accordingly, a deeper incision may not be necessary in some embodiments.

The cutter 106 can be made from one or more of the following materials: polymers, including Nylon, polyamide, polycarbonate (e.g., Makrolon®), acrylonitrile butadiene styrene (ABS), polyester, polyethleneteraphthalate (PET), polyetherethereketone (PEEK™), polyimide, superelastic/shape memory polymers and metals, including spring steel, stainless steel, shape memory metal alloys including nickel titanium alloys (Nitinol), 17-7 PH, cobalt-chromium-nickel alloy (Elgiloy®), and nickel based alloys with chromium and iron (Inconel®). Other appropriate materials can also be used. In embodiments using a “snap-on” feature the frame 200 can be sufficiently flexible to allow the walls of the channel to bend outwardly to accommodate the tubular medical device 108. The slidable cutter 106 can be completely or partially fabricated using one or more of the following methods: casting, laminating, machining, molding (injection or other), sintering, stereo lithography. Other suitable methods can also be used. Advantageously, the slidable tissue cutter 106 can be inexpensive to produce and designed for one-time use. In other embodiments, the tissue cutter 106 can be designed for repeated use following sterilization. An additional advantage of slidable tissue cutter 106 is that it allows for greater precision and ease of use than a hand-held scalpel and is less dependent upon the medical professional's skill and care.
(f) Guided Tissue Dilator

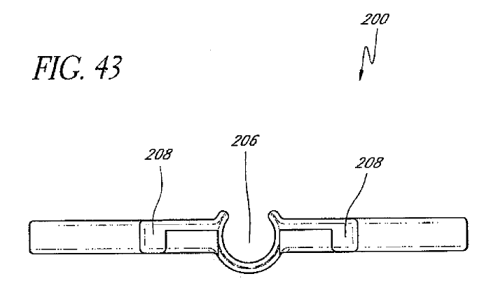

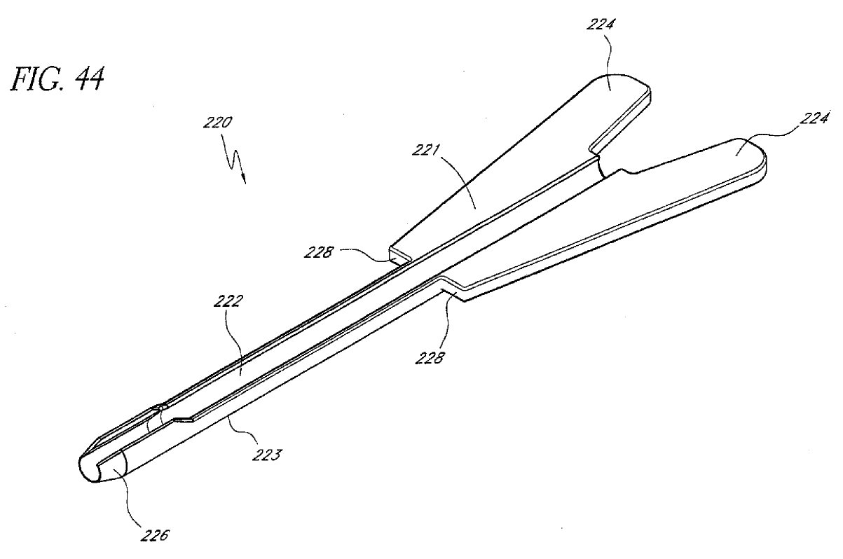

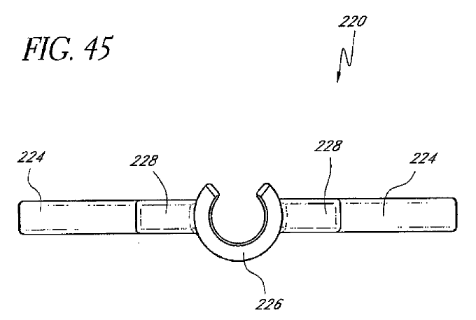

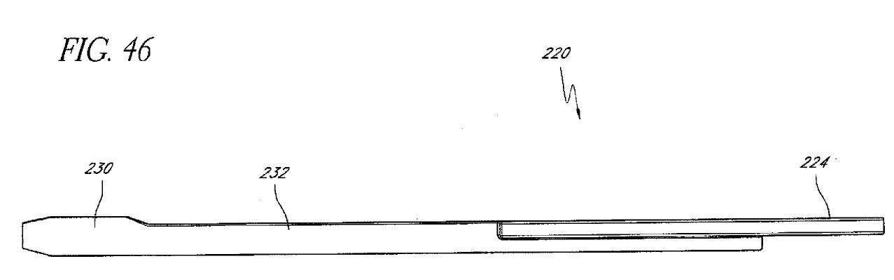

FIGS. 44-46 illustrate an example of a guided slidable tissue dilator 220 which can be used in a vessel closure system 100 in certain embodiments. Tissue dilator 220 can be configured to dilate the tissue tract before the deployment instrument 104 and can be moved through the opening in the skin. Tissue dilator 220 can be generally tube-shaped and configured to snap onto and off of the existing introducer sheath. Dilating the tissue before the advancement of deployment instrument 104 creates a temporary pathway through the tissue, making it easier to advance the deployment instrument 104 forward to the vessel wall 116. After dilating the tissue tract, the tissue dilator 220 is then slid backwards and removed from around the introducer sheath.

Tissue dilator 220 can include an elongate tubular portion 223 with a channel 222. Tubular portion 223 can include a tapered distal end 226 to facilitate insertion of tissue dilator 220 through the percutaneous opening 112. Tissue dilator 220 can include a base 221 with handle portions 224 extending beyond the end of channel 222. As illustrated, surfaces of handles 224 can be positioned in a plane generally parallel to a longitudinal axis of tubular portion 223. In other embodiments, handles 224 can be positioned at an appropriate angle, such as, for example, an angle of at least approximately 90 degree angle. Angled handles can advantageously provide a surface to push on that is perpendicular to the direction of applied force. As with the cutter 106, ends 228 of base 221 can act as mechanical stops to limit the depth of insertion. The medical professional can advance tissue dilator 220 until its distal end 226 encounters the resistance of the vessel wall 116. As with the cutter 106, channel 222 can have a partial circumferential cross-sectional geometry enabling it to “snap on” to an introducer sheath or other medical device. In other embodiments, a tissue dilator can use two mating pieces that clamp or snap together to facilitate temporary attachment and removal. In the illustrated embodiment tubular section 221 includes a distal section 230 and a proximal section 232. Distal section 230 has a greater partial-circumferential cross-section than proximal section 232. In other embodiments, tubular section 221 can be substantially uniform along its length. Tissue dilator 220 can be made from materials and methods similar to those described above with reference to tissue cutter 106.
(g) Additional Clip Structures

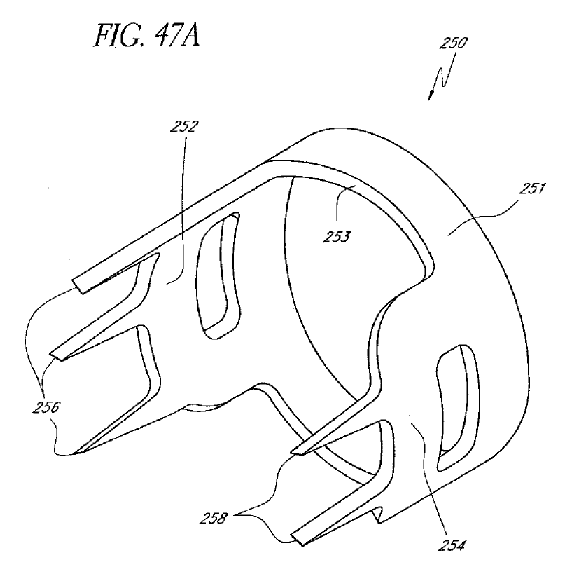

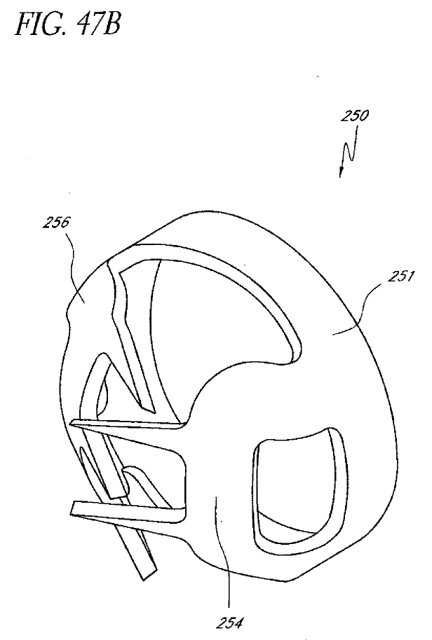

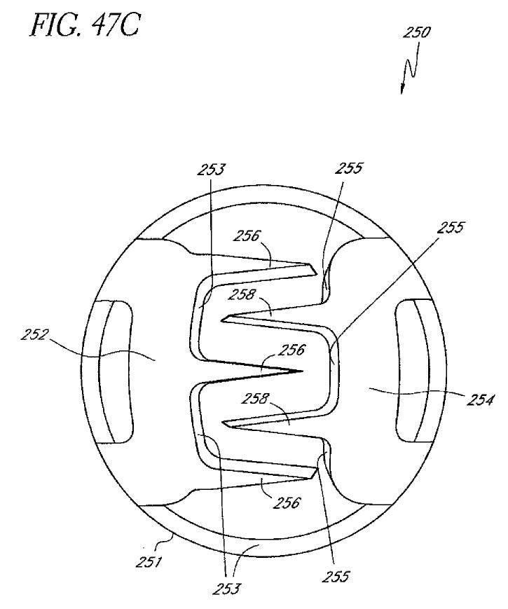

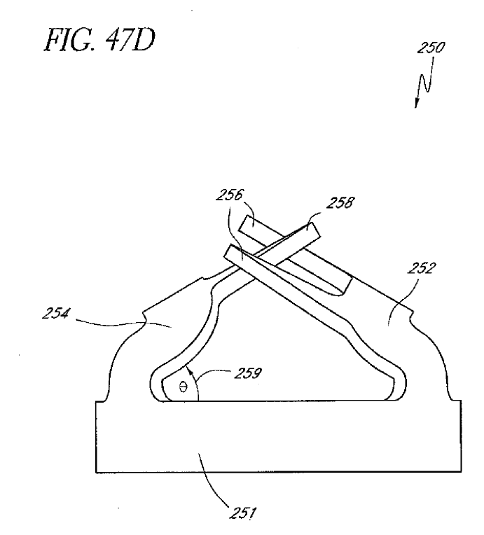

FIGS. 47A-D illustrate another embodiment of a vascular closure clip 250. Clip 250 as illustrated can be similar in many respects to clip 102 except as described below. A primary difference between clip 250 and clip 102 is that the arrangement of fingers 252, 254 on clip 250 is asymmetric: the number of tines 256, 258 on each side is not equal. For example, as illustrated, a first finger 252 can include three tines 256. A second finger 254 can include two tines 258. Tines 256 and tines 258 can be offset from one another and configured to interlace when clip 250 is in a closed configuration as seen in FIGS. 47B-D. For some applications, this interlacing configuration can provide certain advantages over the configuration of clip 102. For example, fingers 252 and 254 can be configured to apply greater compression to tissue and to more completely close the arteriotomy 114, by attempting to draw generally opposing sides of tissue past one another. In addition, the interlaced configuration can in some embodiments, permit a smaller angle θ 259 to be formed between a central axial line or an edge in fingers 252, 254 and a peripheral surface or an edge 253 of a base portion 251 for a given length of fingers 252, 254. In some embodiments, angle θ 259 can be greater than or equal to about 10° and/or less than or equal to about 50°. In some embodiments, angle θ 259 can be about 10°, 15°, 20°, 25°, 30°, 35°, 40°, 45°, or 50°. In a particular example, angle θ 259 can be about 30°. Other suitable angles may also be used. In the illustrated example of FIG. 47C, fingers 252 and 254 do not contact one another when clip 250 is in its closed or deployed configuration. In other embodiments, fingers 252 and 254 can be configured to contact one another in the deployed configuration. For example, tines 258 can be configured to rest on forward surfaces 253 of finger 252. Tines 256 can be configured to rest on forward surfaces 255 of finger 254.

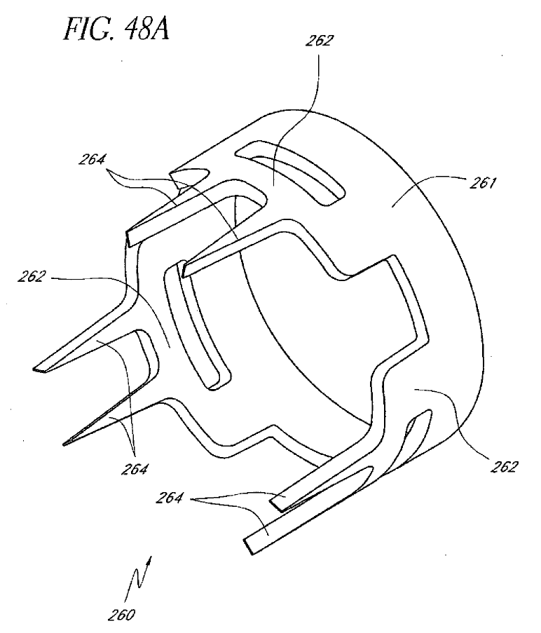

FIGS. 48A-B illustrate another embodiment of a vascular closure clip 260. Clip 260 can be similar to other clips disclosed herein, except as described below. Clip 260 includes three symmetrical fingers 262 extending from annular base 261. As illustrated, fingers 262 can be uniformly spaced around the circumference of base 261. Each finger 262 can include two tines 264. Distal ends of tines 264 are configured to meet when clip 260 is in its closed configuration as illustrated in FIG. 48B.

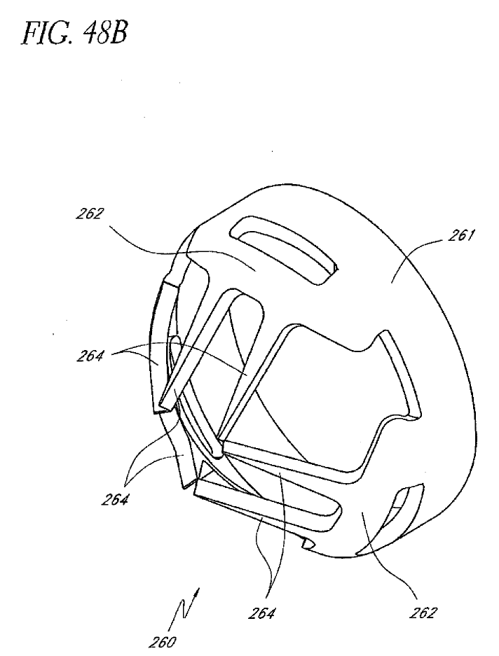

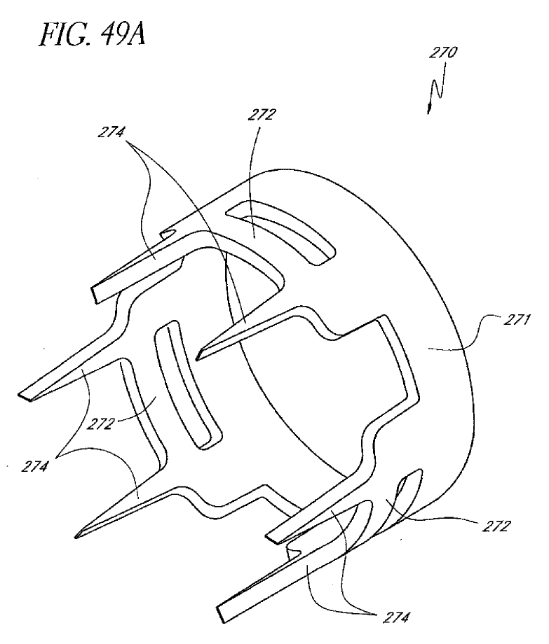

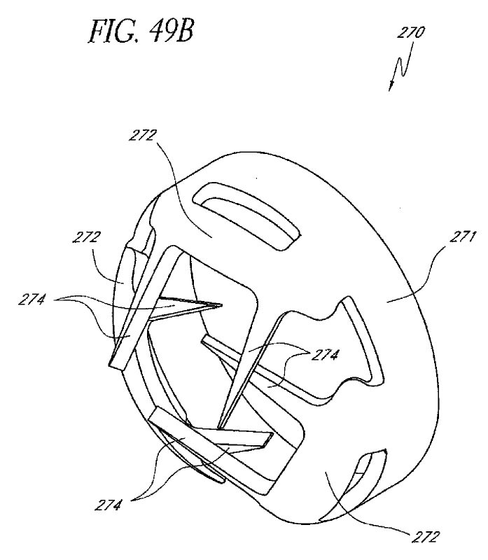

FIGS. 49A-B illustrate another embodiment of a vascular closure clip 270. Clip 270 can include three symmetrical fingers 272 which can be uniformly spaced around the circumference of annular base 271. Clip 270 can be similar in many respects to clip 260. A primary difference between clip 270 and clip 260 is that the tines 274 of clip 270 are configured to overlap tines 274 of adjacent fingers when clip 270 is in a closed configuration.

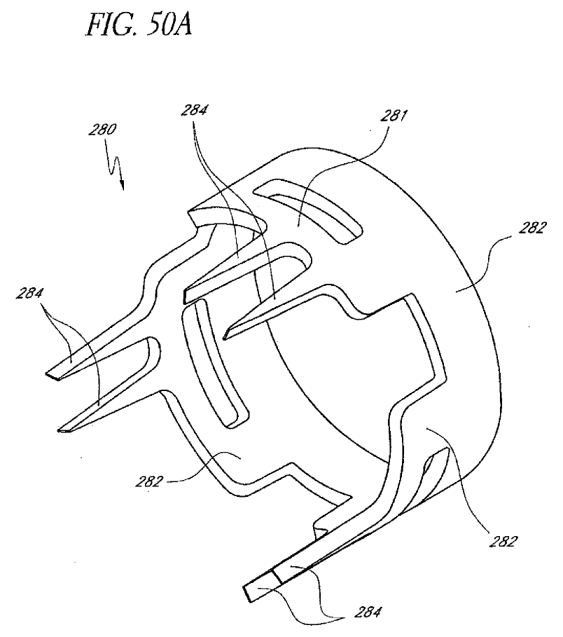

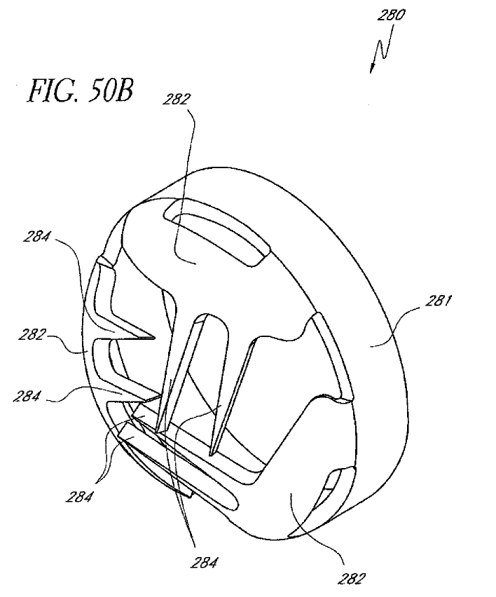

FIGS. 50A-B illustrate another embodiment of a vascular closure clip 280. Clip 280 can be similar to other clips disclosed herein. Clip 280 can include three fingers 284, which can be substantially uniformly spaced around the circumference of annular base 281. Each finger 282 includes two tines 284 which are offset to one side from a central portion of the finger 282. This configuration can permit fingers to bend to a greater degree in the closed configuration without overlapping

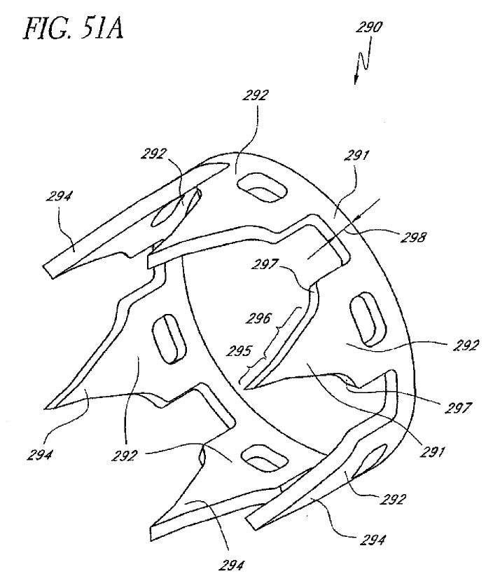

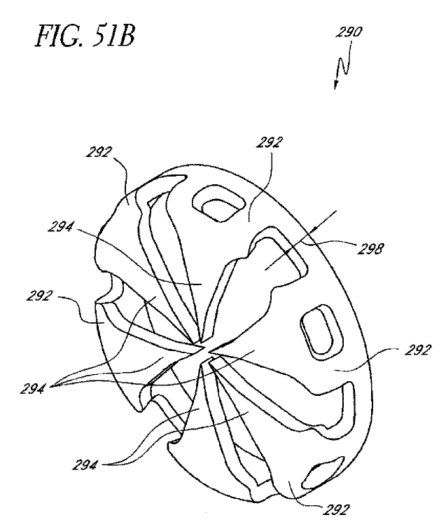

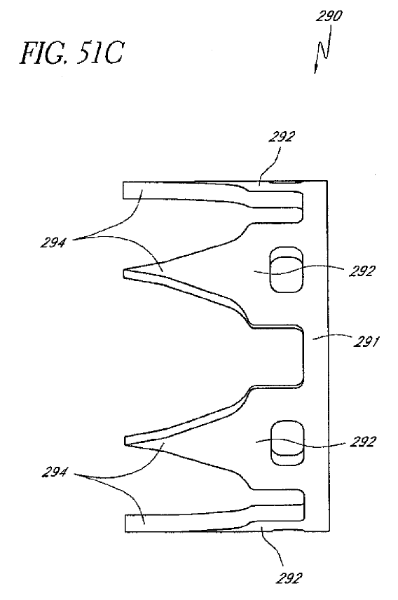

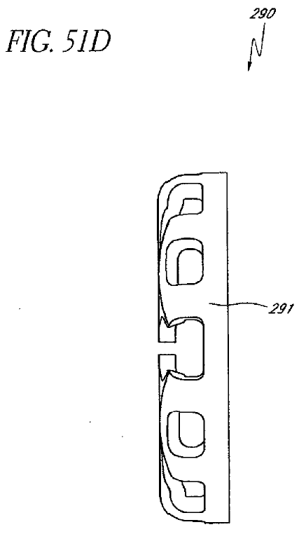

FIGS. 51A-E illustrate another embodiment of a vascular closure clip 290. Clip 290 can be similar to other clips disclosed herein, except as described below. Clip 290 includes six fingers 292 substantially uniformly spaced around a circumference of base portion 291. In some embodiments, each finger 292 includes only a single tine 294. Tines 294 are configured to fold to a substantially flat configuration, best seen in FIG. 49D. Such a configuration permits clip 299 to have a relatively low interior profile. Tines 294 are not configured to contact one another when clip 290 is in a closed configuration. In other embodiments, tines 294 can be configured to meet at or close to a central point, or other point. Tines 294 include a distal-most portion 295 and a second more-proximal portion 296. Portion 295 defines a first interior angle which can be smaller than an interior angle defined by portion 296. Such a configuration gives tines 294 a relatively “sharp” tip and can facilitate the tines' initial penetration of vessel wall 116. Base portion has a height 298. As illustrated height 298 can be relatively small and can be, for example, approximately equal to or less than one fifth of a radius defined by annular base portion 291. A relatively small height 298 permits the clip 290 to have a relatively low external profile when implanted.

Clip 290 can provide more complete circumferential closure by being configured to engage tissue on substantially all sides of arteriotomy. In certain embodiments, it can be more desirable to use such clips 290 for permanent implantation and other clips for temporary implantation. For example, the use of only two opposed fingers can facilitate removal. The use of only two opposed fingers can create a “pinching”-type closing action which can be advantageously simple and predictable.

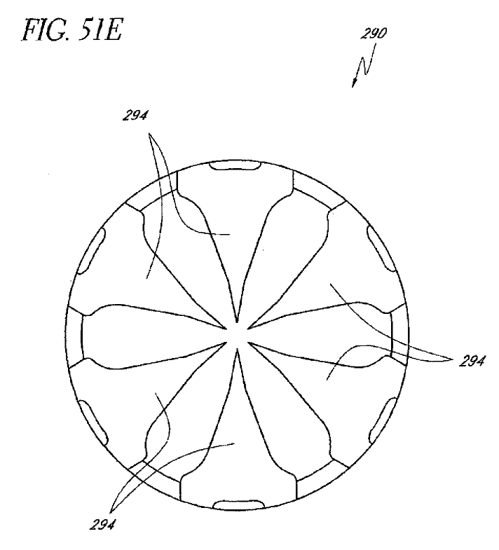

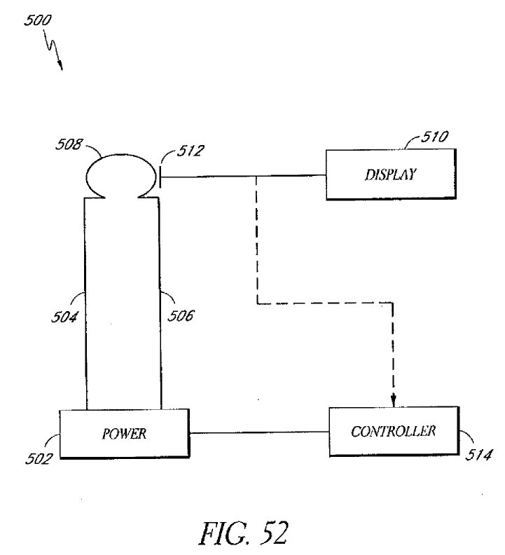

In certain embodiments, heat can be used to facilitate the closure of arteriotomy 114. FIG. 52 illustrates a circuit 500 using direct resistive element heating to heat tissue surrounding the arteriotomy 114. In certain embodiments, selected tissue surrounding the arteriotomy can be heated to a temperature which can be equal to about 40° C., between about 40° C. and 45° C., or greater than about 45° C. In certain embodiments, selected tissue surrounding the arteriotomy can be heated to a temperature of about 35° C., 36° C., 37° C., 38° C., 39° C., 40° C., 41° C., 42° C., 43° C., 44° C., 45° C., 46° C., 47° C., 48° C., 49° C., or 50° C. Other suitable temperatures may also be used. At these temperatures, tissue being compressed together by a vascular closure clip can undergo cellular changes that tend to fuse tissue together to close the arteriotomy.
(h) Heated Systems

Heat can be used with any of the vascular closure clips described above, such as, for example clip 102. A power source 502 such as an RF power source is provided. Other suitable power sources such as a DC power source can be used. Power source 502 is connected to a resistive element 508 via conductors 504 and 506. Clip 102 can function as the circuit's resistive element 508. In certain embodiments, only a portion of clip 102 will function as the resistive element. Clip 102 can be treated to increase its resistance value by, for example, being covered with a resistive coating. An increased resistance can reduce the power level necessary to effectuate a given amount of heating. In certain embodiments, portions of the clip 102 are covered with a thermally and/or electrically insulative coating. The remaining, uncovered portions of clip 102 can be configured to transfer thermal energy to the tissue being heated. In certain embodiments, only the tines or a distal portion of the tines are configured to transfer the thermal energy to the tissue. Conductors 504 and 506 can include wires made from a suitable electrically-conductive material such as copper-clad steel. In certain embodiments, conductors 504 and 506 can also function as tethering elements to allow removal of clip 102. Conductors 504 and 506 can be covered with an insulating cover or coating. A thermocouple 512 can be mounted to the clip to monitor the temperature of the clip and/or the surrounding tissue. The recorded temperature can be provided to a user display 510 and/or controller 514. Controller 514 permits the medical professional to adjust the amount of power delivered to the resistive element 508. In certain embodiments, the power delivered can be less than about 2 W, between about 2 and about 50 W, or greater than 50 W. In certain embodiments, the power delivered can be about 2 W,3 W, 4 W, 5 W, 6 W, 7 W, 8 W, 9 W, 10 W, 11 W, 12 W, 13 W, 14 W, 15 W, 16 W, 17 W, 18 W, 19 W, 20 W, 21 W, 22 W, 23 W, 24 W, 25 W, 26 W, 27 W, 28 W, 29 W, 30 W, 31 W, 32 W, 33 W, 34 W, 35 W, 36 W, 37 W, 38 W, 39 W, 40 W, 41 W, 42 W, 43 W, 44 W, 45 W, 46 W, 47 W, 48 W, 49 W, or 50 W. Other suitable wattages may also be used. The medical professional can maintain the tissue at the desired temperature for a certain length of time. In some embodiments, heat can be applied to the tissue for a period less than or equal to about 30 seconds, or greater than 30 seconds. Other suitable times may also be used.

Following the application of heat, the conductors 504, 506 can be disconnected from clip 102 in many ways. For example, a twisting, cutting, or other manipulative action can be used to remove the conductors. In embodiments using temporary or removable clips, conductors 504, 506 can be used as a primary or backup tethering element to remove the clip 102 following hemostasis. In certain embodiments, conductors 504, 506 can be connected to the clip 102 via spot welding, mechanical fit, soldering, combination, or other suitable method. Conductors 504, 506 can be fabricated from many different materials, such as copper, platinum, stainless steel, or a composite of materials (e.g. copper clad steel or platinum and silver combined by a drawn filled tubing process). In certain embodiments, conductors 504, 506 can include composite signal wires using silver as the inner core to better transmit, for example, radiofrequency or direct current energy. Conductors 504, 506 can be fabricated with a circular, elliptical, rectangular (flat), or other geometry which may depend on the space available on the clip 102. Conductors 504, 506 can be covered or jacketed with an insulative material such as polyimide, polyamide, polyurethane, polyester, nylon, or other suitable material.

In certain embodiments, a special tip can be placed over a standard electrosurgical tool such as, e.g., a Bovie Instrument (i.e., digital electrosurgical generator and accessories by Bovie Medical Corporation), to insert through the skin and make contact with the closure device and/or tissue. In certain embodiments, alternative heating means can be provided to heat the clip and/or the adjacent tissues including, for example, ultrasound energy, microwave energy, etc.

FIG. 53 illustrates a circuit using ohmic tissue beating to heat tissue. A power source 502 such as a radiofrequency (RF) or direct current (DC) power source is provided. Power source 502 is connected to an active electrode 524 via conductor 526. Clip 102 can function as the active electrode 524. Alternatively, only a portion of clip 102 can function as the active electrode 524. For example, in certain embodiments, one or more of the clip's tines or only a portion of the clip's tines such as the distal-most portion can function as the active electrode 524. In certain embodiments, remaining portions of the clip 524 are covered with an electrically insulating cover or coating. A second conductor 528 connects power source 502 to an indifferent electrode 522. The indifferent electrode 522 can be, for example, an electrode plate or large surface area indifferent ground pad applied to the patients skin. The indifferent electrode 522 can be placed on the patient's back, thigh or other location. The indifferent electrode 522 can be applied to a portion of the patient's skin generally opposite the percutaneous opening. The power supply 502 applies a voltage differential across the active and indifferent electrodes 524, 522 causing current to flow through the intervening tissue thus heating the tissue. The heat is generally concentrated at tissue adjacent to the active electrode 524. Controller 514 can permit the medical professional to adjust the amount of power delivered.

In another embodiment (not shown), a first portion of the clip can act as a first electrode and a second portion of the clip can act as a second electrode. The first and second portions of the clip can be electrically insulated from one another. For example, a first finger or a portion of the first finger such as one or more tines can act as the first electrode and a second finger or a portion of the second finger can act as the second electrode. A power source applies a voltage differential across the first and second electrodes causing current to flow between them and heat intervening tissue.

An electrode-enabled closure device can also be used to confirm contact between the closure device and the tissue surface, such as by comparing the impedance between an electrode element and a return path (indifferent electrode or second electrode). When an electrode surface contacts only or primarily blood, the measured impedance can be substantially higher than when a small or substantial portion of the electrode surface contacts tissue.

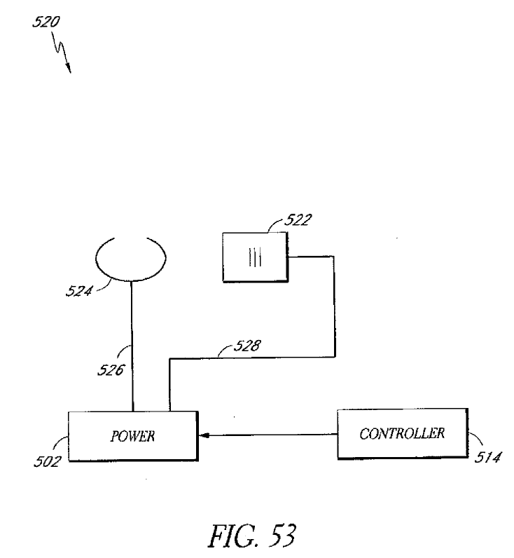

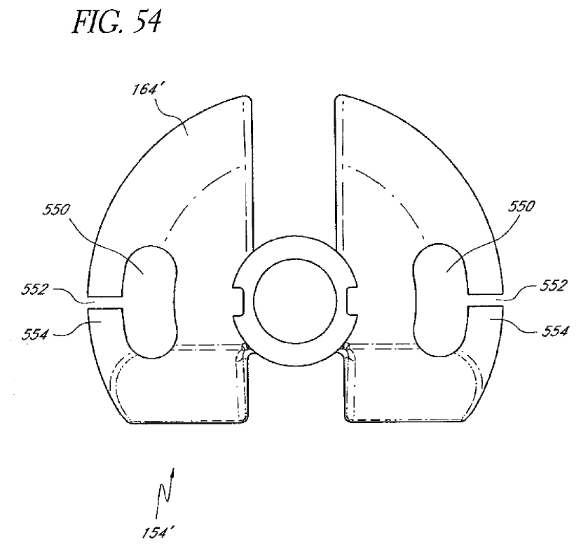

FIGS. 54-55 show another embodiment of an inner tubular member 154′ which can form one component of a deployment instrument. Inner tubular member 154′ can be similar to inner tubular member 154 described above. A primary difference between inner tubular member 154′ and inner tubular member 154 is the inclusion of recessed portions 550 on handle 164′. Connecting channels 552 can be relatively thin and can permit access from an exterior surface of handle 164 to an interior of recessed portions 550. Recessed portions 550 can receive the proximal ends of suture lines 234. For example, the proximal ends of suture lines 234 can be tied to or looped around portions 554 of handle 164′. A removable clip can be implanted using the procedure described above. Prior to removal of the deployment instrument, the suture lines 234 can be removed from portions 554 of handle 164′. Following hemostasis, the proximal ends of the suture lines 234 can be grasped to withdraw the clip from the vessel and out of the patient.
(i) Clip Variations and Manufacture

Other clip variations are also possible. The tissue compression can be modified by adjusting one or more of several tissue engagement element design attributes, such as the length, width, thickness, angle, number and location of the elements, etc. The proximal edge of the clip can have a straight, sinusoidal, notched, keyed, combination or other suitable design. The proximal edge geometry can mate with a contacting surface of the advancement and deployment instrument. Clips can be made from one or more of a tubing, sheet, wire, strip, band, rod, combination or other suitable material.

In certain embodiments, the clip can be configured to be in its malleable martensite phase at room temperature. The clip can be loaded onto a deployment instrument in an open configuration. The clip can be configured to transition to an austentite phase by the application of heat during or after deployment. The application of heat can cause the clip to revert to its memorized, closed configuration. In certain embodiments, the clip can be configured to revert to its closed configuration upon being heated to a temperature near the temperature of the human body. In such embodiments, the clip can be delivered to the arteriotomy and partially deployed or held in place on the exterior of the vessel wall 116 for a period of time sufficient to heat the clip to its austentite transition temperature. In other embodiments, heat may be applied via insertion of a heated probe or remotely via application of focused electromagnetic energy.

The clip can include at least one (single element) hinge feature to assist with deployment, tissue engagement, compression and or removal from the tissue. The clip can be partially or completely made from one or more of the following materials: superelastic/shape memory polymers, metals including, spring steel and stainless steel, metal alloys including nitinol, 17-7 PH, Elgiloy, and Inconel. Other appropriate materials can also be used. In a preferred embodiment, the clip can be partially or completely made from a superelastic and/or shape memory material such as nitinol. A discussion of certain properties of superelastic and/or shape memory materials can be found in U.S. Pat. No. 7,182,771, the entirety of which is hereby incorporated by reference herein and made a part of the present specification. In certain embodiments, such as those using nitinol or other superelastic and/or shape memory materials, it can be desirable for the clip to have a relatively tight bend in a memorized configuration. In some circumstances, it can be advantageous to use a bend sufficiently tight that it would normally exceed the elastic limit of the material and thus permanently deform it. To prevent permanent deformation, a bend can be produced in the device followed by an annealing process to relieve bending stresses within the device. Following this first bend, the device can be bent further to produce an even sharper bend, and then re-annealed to alleviate the stress from this additional bending. This process can be repeated to attain a desired substantial bend, or reduced radii, or reduced angle that would otherwise permanently deform the device if the bend were attempted in a single bending event. In certain embodiments, any surface of the clip that comes in contact with blood and/or tissue can he electropolished, especially metal or metal alloy surfaces, such as a superelastic/shape memory alloy. Electropolishing may be used to produce smooth surfaces. Electropolishing can also beneficially remove or reduces flash and other artifacts from the fabrication of the device.

The clip can have a completely contiguous cross section, or partial, incomplete contiguous cross section. A discontiguous cross-section can permit certain embodiments of the clips to be loaded from the side of the vascular introducer and/or deployment instrument. In certain embodiments, the deployment instrument can include a slot or opening permitting the deployment instrument to be secured to the tubular medical device from the side. Tissue engagement elements (e.g., tines, fingers, protrusions, etc.) can be parallel, overlapping, crossing, spiral, combination or other. The clip can include tissue engagement elements with the same, different or combination lengths. The clip can compress tissue on a horizontal plane, vertical plane or a combination of both. The tissue engagement elements can be straight, curved or a combination of both. The tissue attachment motion/direction can be straight, twisted, rotated, combination or other suitable and desirable motion or motions.
(j) Swellable Plugs

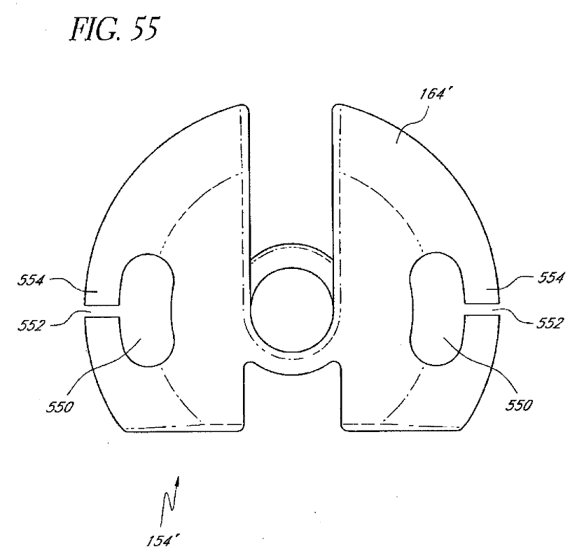

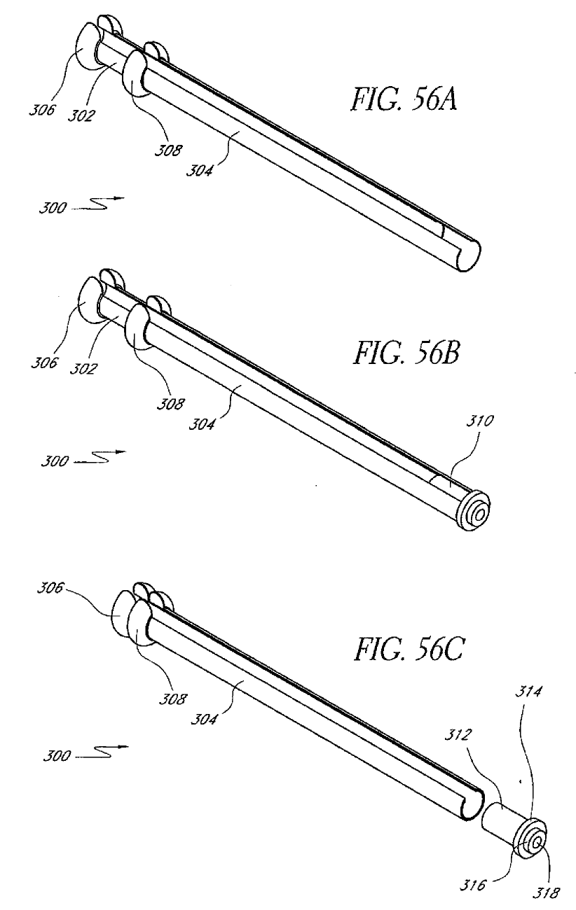

FIGS. 56A-C illustrate an additional embodiment of a vessel closure system. In one embodiment, a swellable plug 310 which can be bioabsorbable is loaded onto the distal end of a plug deployment instrument 300. Plug deployment instrument 300 can include an inner tubular member 302 with handle 306 and an outer tubular member 304 with handle 308. The proximal end 312 of plug 310 can be received by the distal end of outer tubular member 304. Intermediate stop portion 314 of plug 310 can have a larger outer diameter than either proximal end 312 or distal end 316 and is received against the distal end of the outer tubular member 304. As illustrated, stop portion 314 can have a generally circular geometry. However, other suitable shapes or geometries can be used. For example, in certain embodiments stop portion 314 can have a flared or tapered shape, a general ‘X’ shape, an inverted general ‘T’ shape, a combination or any other suitable shape or geometry. In certain embodiments, stop portion 314 can be slotted or ribbed to facilitate flexing during advancement. Proximal end 312 can be relatively long to facilitate plug kinking as will be described below. In certain embodiments, proximal end 312 can have a length that is greater than or equal to about twice the length of distal end 316, and/or greater than or equal to about five times the length of distal end 316. Plug 310 can include a longitudinal channel 318 allowing the deployment instrument 300 and plug 310 to be advanced over a tubular medical device in a similar fashion to that described above with respect to deployment instrument 104. Inner tubular member 306 can be advanced distally by applying pressure to handle 306 and/or by pulling handle 308 in a proximal direction. A stop means such as a removable element affixed to the outer tubular member 302 between handles 306 and 308 can maintain separation of handles 306 and 308 until the medical professional is ready to begin deployment. Once the medical professional has confirmed proper placement of the distal end of the deployment instrument 300, the stop means can be overcome by for example removing the removable element in order to begin deployment. The distal end of inner tubular member 306 pushes plug 310 free of the outer tubular member 304 to effect deployment. The deployment instrument 300 can be configured such that the plug 310 will be fully deployed when the handles 306 and 308 have been brought together.

Swellable plug 310 can be partially or completely fabricated from materials that swell or expand when they are exposed to a fluid, such as blood or subcutaneous fluid, or another fluid, for example, that can be added by the physician to cause the material to swell. These materials include hydrophilic gels (hydro gels), regenerated cellulose, polyethylene vinyl acetate (PEVA), as well as composites and combinations thereof and combinations of other biocompatible swellable or expandable materials. Upon deployment, swellable plug 310 can swell causing longitudinal channel 318 to be occluded and sealing the arteriotomy. In certain embodiments, plug 310 can be partially or completely fabricated from a lyophilized hydrogel, such as, for example polyethylene gycol (PEG) or other polymer carrier. The polymer used in the carrier can include hydrolytically degradable chemical groups, thereby permitting in vivo degradation. Hydrophilic polymeric materials suitable for use in forming hydrogels include poly(hydroxyalkyl methacrylate), poly(electrolyte complexes), poly(vinylacetate) cross-linked with hydrolysable bonds, water-swellable N-vinyl lactams polyscaccharides, natural gum, agar, agarose, sodium alginate, carrageenan, fucoidan, furcellaran, laminaran, hypnea, eucheuma, gum Arabic, gum ghatti, gum karaya, gum tragacanth, locust beam gum, arabinogalactan, pectin, amylopectin, gelatin, hydrophilic colloids such as carboxymethyl cellulose gum or alginate gum crosslinked with a polyol such as propylene glycol, and the like. Several formulations of previously known hydrogels are described in U.S. Pat. No. 3,640,741 to Etes, U.S. Pat. No. 3,865,108 to Hartop, U.S. Pat. No. 3,992,562 to Denzinger et al., U.S. Pat. No. 4,002,172 to Manning et al., U.S. Pat. No. 4,014,335 to Arnold, U.S. Pat. No. 4,207,893 to Michaels, and in Handbook of Common Polymers, (Scott and Roff, Eds.) Chemical Rubber Company, Cleveland, Ohio, all of which disclosures in the foregoing patents and publication regarding hydrogels are incorporated herein by reference.

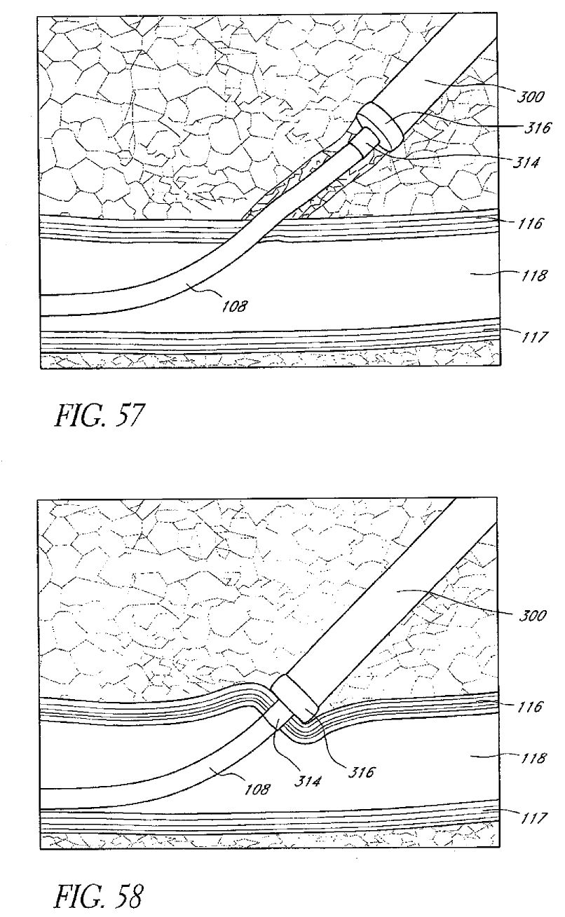

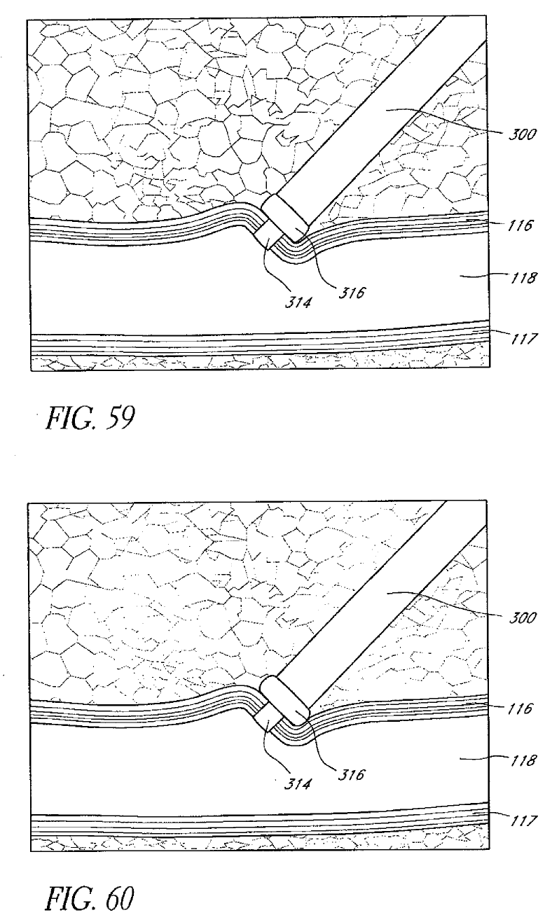

An example of a method for using plug deployment instrument 300 and plug 310 will now be described with reference to FIGS. 57-63. The deployment instrument 300 loaded with plug 310 can be advanced over a previously installed tubular medical device 108 as shown in FIG. 57 until the distal end 316 encounters vessel wall 116. In certain embodiments, as illustrated, distal end 316 can be received within the arteriotomy 114. In other embodiments, distal end 316 can be received against an outer surface of vessel wall 316. Intermediate portion 314 can be configured to act as a stop to prevent overinsertion of plug 310 into the vessel. The introducer sheath can then be removed from the vessel as shown in FIG. 59.

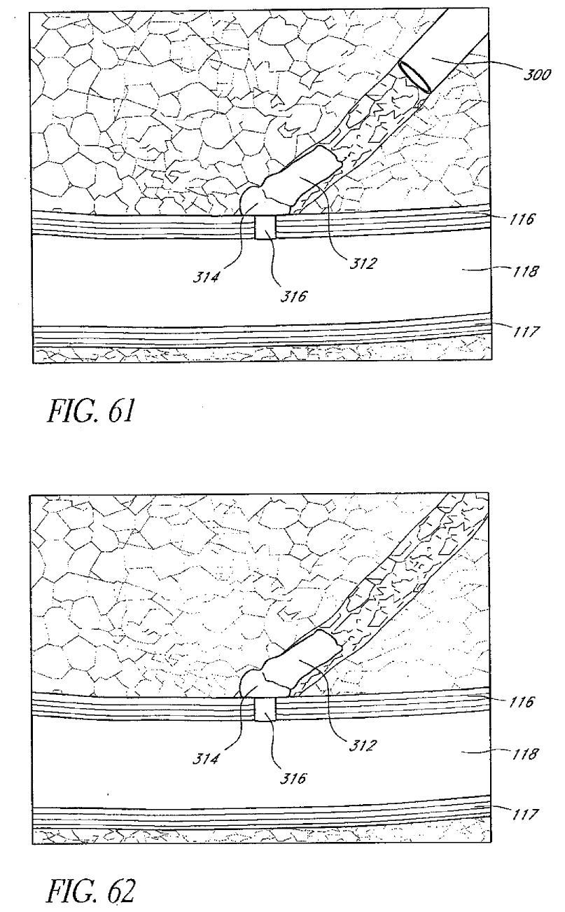

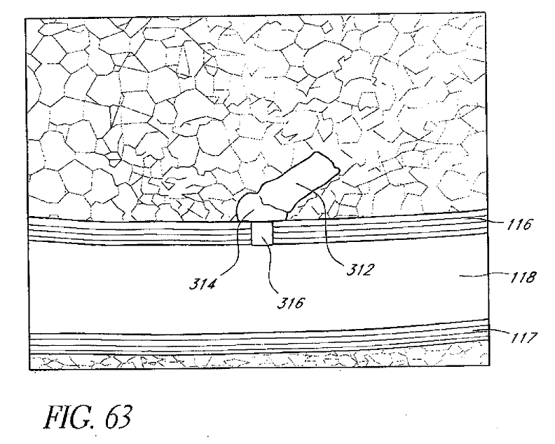

As shown in FIG. 60, the deployment instrument 300 can be held in place against the vessel wall 316 while the exposed portions of plug 310 begin to swell. The swelling can be initiated or accelerated by various events, such as coming into contact with blood and/or subcutaneous fluid. In certain embodiments, the enlargement of distal end 316 can help to secure the plug 310 in place within the arteriotomy 114. The swelling of plug 310 can occlude longitudinal channel 318, tending to seal or otherwise partially or entirely fill the arteriotomy 114. Alternatively or additionally, the channel 318 can be occluded via kinking of proximal portion 312. Once the plug 310 is secured to the vessel wall 116, deployment instrument 300 can be removed as shown in FIG. 61. Fatty tissue that was previously displaced by the deployment instrument 300 may begin to fill in the tissue tract. This tissue can thus apply pressure to proximal portion 312 tending to kink or occlude it. Patient movement and/or externally or internally applied pressure can also be used to cause the proximal portion 312 to kink. Deployment of plug 310 at an acute angle to the vessel wall, as illustrated, can also increase the tendency of proximal portion 312 to kink. In certain embodiments, the inner surface of the longitudinal channel 318 can be configured to stick to itself when one region of it contacts another region. For example, in certain embodiments inner surfaces of longitudinal channel 318 can be coated with an adhesive or other appropriate coating to assist in occluding the longitudinal channel 318. In certain embodiments, the adhesive or coating can be configured to avoid or to diminish adherence to the deployment instrument 300. FIG. 62 shows an embodiment of the deployed plug 310 in a fully swollen state. Plug 310 can be completely or partially bioabsorbable. In certain embodiments, plug 310 can be configured to be completely absorbed by the patient's body after about 4 weeks. Other suitable times may also be used. FIG. 63 shows the plug 310 in a partially-dissolved state.

Swellable plug 310 can be shielded from unintended contact with fluid (blood, saline, etc.), before insertion into the body, by a removable wrapper or dissolvable coating. Swellable plug 310 can include a relatively rigid outer coating that begins to dissolve upon exposure to fluids such as blood, thus providing time for the medical professional to position the plug 310 within the arteriotomy. In some embodiments, a plug can be configured to be advanced directly over the tubular medical device 108 and deployment instrument 310 can be replaced with a pusher instrument. In certain embodiments, a plug can include a longitudinal slit or spiral allowing the plug to be attached to the tubular medical device or deployment instrument from the side. In certain embodiments, the deployment instrument can also include a slot allowing attachment from the side.

The vascular closure device can incorporate one or more coatings, materials, compounds, substances, drugs, therapeutic agents, etc., that positively affect healing at the site, at and or near where the device is deployed, either incorporated into the structure forming the device, incorporated into a coating, or both. Thromboresistance materials, antiproliferative materials, or other coatings intended to prevent thrombosis (acute and or chronic), hyperplasia, platelet aggregation, or other negative response, at or near the attachment of the device within the body. The coatings, materials, compounds, substances, drugs, therapeutic agents, etc., can be used by themselves, and/or contained in a carrier such as a polymeric matrix, starch, or other suitable material or method. The coatings can be liquid, gel, film, uncured, partially cured, cured, combination or other suitable form.
(k) Delivery Features

Many different types of delivery features, such as coatings on the vascular closure device, can be used to deliver therapeutic agents, including (but are not limited to) one or more of the following: antiproliferative/antimitotic agents including natural products such as vinca alkaloids (i.e. vinblastine, vincristine, and vinorelbine), paclitaxel, epidipodophyllotoxins (i.e. etoposide, teniposide), antibiotics (dactinomycin (actinomycin D) daunombicin, doxorubicin and idarubicin), anthracyclines, mitoxantrone, bleomycins, plicamycin (mithramycin) and mitomycin, enzymes (L-asparaginase which systemically metabolizes L-asparagine and deprives cells which do not have the capacity to synthesize their own asparagine); antiplatelet agents such as G(GP) II.sub.b/III.sub.a inhibitors and vitronectin receptor antagonists; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nirtosoureas (carmustine (BCNU) and analogs, streptozocin), trazenes—dacarbazinine (DTIC); antiproliferative/antimitotic antimetabolites such as folic acid analogs (methotrexate), pyrimidine analogs (fluorouracil, floxuridine, and cytarabine), purine analogs and related inhibitors (mercaptopurine, thioguanine, pentostatin and 2-chlorodeoxyadenosine {cladribine}); platinum coordination complexes (cisplatin, carboplatin), procarbazine, hydroxyurea, mitotane, aminoglutethimide; hormones (i.e. estrogen); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory; antisecretory (breveldin); anti-inflammatory: such as adrenocortical steroids (cortisol, cortisone, fludrocortisones, prednisone, prednisolone, 6.alpha.-methylprednisolone, triamcinolone, betamethasone, and dexamethasone), non-steroidal agents (salicylic acid derivatives i.e. aspirin; para-aminophenol derivatives i.e. acetominophen; indole and indene acetic acids (indomethacin, sulindac, and etodalac), heteroaryl acetic acids (tolmetin, diclofenac, and ketorolac), arylpropionic acids (ibuprofen and derivatives), anthranilic acids (mefenaric acid, and meclofenamic acid), enolic acids (piroxicam, tenoxicam, phenylbutazone, and oxyphenthatrazone), nabumetone, gold compounds (auranofin, aurothioglucose, gold sodium thiomalate); immunosuppressives: (cyclosporine, tacrolimus (FK-506), sirolimus (rapamycin), azathioprine, mycophenolate mofetil); angiogenic agents: vascular endothelial growth factor (VEGF), fibroblast growth factor (FGF); angiotensin receptor blockers; nitric oxide donors; anti-sense oligionucleotides and combinations thereof; cell cycle inhibitors, mTOR inhibitors, and/or growth factor signal transduction kinase inhibitors. Alternatively, a clot promoter can be used, such as protamine sulphate or calcium hydroxide. Endothelial cells can also be added to the vascular closure device.

One or more of the therapeutic agents can be included in the device in many ways, such as by blending them into the device base materials during fabrication, applying them just prior to deployment, or applying them after the device has been deployed. One or more therapeutic agents can be used on a single device. The delivery feature can be designed to provide benefits rapidly or over an extended period of time. The delivery feature can be stable or eluting. The coatings, materials, compounds, substances, therapeutic agents, etc., can elute from a coated (or embedded) device (or component) over time and enter the surrounding tissue. In certain embodiments, the delivery feature can be effective during a period of at least about three days in some applications, between about seven and about thirty days in other application, and/or up to approximately six months in some applications.
(l) Fabrication Alternatives

Post device fabrication coating methods can include, but are not limited to, spin coating, RF-plasma polymerization, dipping, spraying, brushing, submerging the devices into a beaker containing a therapeutic solution while inside a vacuum chamber to permeate the device material, etc.

Alternatively, or in combination with the above therapeutic substances, one or more materials such as platinum, gold, tantalum, tin, tin-indium, zirconium zirconium alloy, zirconium oxide, zirconium nitrate, phosphatidyl-choline, pyrolytic carbon, combination or other material, can be deposited onto the closure device surface using electroplating, sputtering vacuum evaporation, ion assisted beam deposition, vapor deposition, silver doping, boronation techniques, or other coating process.

Radiopaque material such as barium sulfate, bismuth trioxide, tantalum, platinum/iridium or other suitable materials can be added to any of the closure devices for enhanced visualization under a fluoroscope or other visualization means commonly used in a catheterization lab or surgical suite. Additionally, such materials can be added to the closure device by sputter coating, ion deposition, vapor deposition, combination, or other suitable processes.
(m) Additional Uses

In certain embodiments, the distal end of inner tubular member can have at least one section with a larger circumferential diameter or flare to cause clip tines to deflect outward (during forward movement during deployment), capturing more tissue (than without the increased diameter section) as the clip is advanced forward, for greater tissue compression and sealing. The distal end of the inner tubular member can also have a non circumferential enlargement such as at least one bump or raised surface arranged around the circumference. This design can be used to cause only some of the clip tines to be opened or deflected outward during advancement and deployment, or some to deflect more than others.

In certain embodiments, the deployment instrument can be configured so that the clip is deployed by advancing the outer tubular member distally relative to the inner tubular member instead of by proximally withdrawing the inner tubular member. The pressure element or other pressure sensing means can be secured to the inner tubular member, such as for example at a proximal end of the inner tubular member.

In certain embodiments, suction can be used to temporarily attach the deployment instrument to the vessel wall, and/or to confirm contact with the desired tissue. The deployment instrument can be configured to enable local and/or remote suction. In certain embodiments, an elongate suction tube or lumen can be secured to and/or located within the deployment instrument. The suction tube can include an opening on or near the distal end of the deployment instrument, and a valve or fitting (such as, for example, a Luer fitting) on the side or proximal end of the tool, to which a syringe, bulb, or other suction device could be attached and/or integrally formed. In certain embodiments, local suction can be accomplished without attachment to an external vacuum source. Local suction can be accomplished, for example, using a syringe or other physician manipulated device to pull a vacuum, creating the desired suction. A Luer-lock or stopcock then can be used to close the suction tube or lumen containing the vacuum to maintain a suction condition. In certain embodiments, a remote vacuum suction system can be attached to a vacuum line. The vacuum system can include a means for limiting the amount of vacuum/suction which can be created in order to prevent trauma to the tissue adjacent to the distal suction port.

The slidable tissue cutter can be adapted to use heat to cut skin and or other tissue by making the leading edge an electrode and attaching at least one electrical conductor to the electrode. Direct resistive element heating or ohmic tissue heating can be used. Biocompatible materials (e.g., gold, platinum, platinum/iridium, stainless steel, nitinol and other suitable materials) can be used for the electrode and connected to a suitable (e.g., electrical and biocompatible) conductor. For ohmic tissue heating, one conductor can be connected to an RF power source. Another conductor is connected to a ground pad placed on the patients body, and also connected to the power source. For direct resistive element heating, both conductors from the power source are connected to an electrode.

In certain embodiments, the cutting elements of slidable tissue cutter can be designed to cut tissue or to both cut and remove tissue. In some cut-and-remove embodiments, the cutting element can be circular, diagonal, angled, or other blade. The slidable tissue cutter can be designed and used to cut any body tissue including, but not limited to, skin, fat ligaments, cartilage, bone, or vessels. The cutting element can be of any desirable type, including thermal (laser, RF, etc.), chemical, ultrasonic, combination, or other.

This disclosure has provided certain examples of closure devices including clips and plugs. However, other types of closure devices can be used. In certain embodiments, a closure device can be smaller in an initial configuration or in a deployed configuration. In certain embodiments, the closure device can close a tissue opening by bringing closer together sides of the tissue opening and/or by partially or completely occluding the opening. The closure device can be partially or completely made from one or more of a polymer, rubber, silicone, metal, metal alloy, superelastic/shape memory polymers and metallic alloys, or other suitable material or materials.

In some embodiments, the closure device may be partially or completely fabricated from a biodegradable/bioabsorbable material, including but not limited to one or more of starch, modified cellulose, collagen, fibrin, fibrinogen, fibronectin, elastin, vitronectin, laminin, thrombin, albumin and gelatin or other connective proteins or natural materials, polymers or copolymers such as polyvinyl pyrrolidone, polylactide [poly-L-lactide (PLLA), poly-D-lactide (PDLA)], polyglycolide, polydioxanone, polycaprolactone, polygluconate, polylactic acid (PLA), polylactic acid-polyethylene oxide copolymers, poly(hydroxybutyrate), polyanhydride, polyphosphoester, poly(amino acids), poly(alpha-hydroxy acid) poly d,l-actic acid (PLA) and copolymers of lactic acid and glycolic acid (PLGA), or related copolymers of these materials as well as composites and combinations thereof and combinations of other biodegradable/bioabsorbable materials. In some embodiments, the closure device can be partially or completely fabricated from a biocompatible material, such as expanded polytetrafluoroethylene (ePTFE), polyester, polyurethane, silicone, rubber, Dacron, and/or urethane.

In some embodiments, the closure device can include one or more coatings and/or be partially or completely formed from one or more of the following: swellable materials, bioabsorbable materials, and biocompatible materials.

In some embodiments, the closure device can have a bicompatible contact surface such as adhesives, bonding compounds, or other solutions, including those intended to delay swelling or expansion of at least one section of the closure device once it comes in contact with a fluid. The biocompatible contact surface can be located on any surface or all surfaces of the closure device. The contact surface can be applied or integrated into the device in many ways, such as during the manufacturing process, just prior to deployment, or after the device has been deployed. The bonding materials can be in the form of a liquid, semi solid, or solid. Suitable bonding materials can include gels, foams and microporous mesh. Suitable adhesives can include acrylates, cyanoacrylates, epoxies, fibrin-based adhesives, other biological based adhesives, UV light and/or heat activated or other specialized adhesives. The contact surface can bond on initial contact, or after a longer period of time to allow repositioning of the closure device if desired. Such a contact surface can include a crystalline polymer that changes from a non-tacky crystalline state to an adhesive gel state, such as when the temperature is raised from room temperature to body temperature. An example of such material is available under the trade name Intillemer™ adhesive, available from Landec Corp., as well as composites and combinations thereof and combinations of other materials. Suppliers of biocompatible adhesives include, but are not limited to, Plasto (Dijon, France), Haemnacure (Montreal, Canada), Cohesion (Palo Alto, Calif.), Cryolife (Kennesaw, Ga.), TissueLink (Dover, N.H.), and others. To increase the work time of the contact surface and/or to allow repositioning of the closure device after it has been deployed, the contact surface can be blended with a material such as a starch or other material, that retards or delays bonding to allow repositioning of the device after it has been deployed. A degradable coating can be placed over the contact surface so that it degrades and exposes the adhesive. Other contact surfaces can include composites-based adherents and combinations of the above materials and other suitable materials as are known in the art.

The closure devices, systems, and methods can be used for any suitable cardiovascular, gastrointestinal, neurological, reproductive, lymphatic, respiratory, orthopedic, or other applications where partial or complete, temporary, removable, or permanent closure, compression, sealing, bringing together, cinching, anchoring, and/or reinforcement is desired. Additionally, the closure devices, systems, and methods can be used in connection with any lumen, duct, organ, hollow body organ or cavity, or other bodily structures or tissues, where partial or complete, temporary, removable, or permanent sealing, crimping, compression, plugging, reinforcement or other purpose is desired. For example, some applications include, but are not limited to, the following: cerebral aneurysm treatment, shortening the chordae tendinae to treat mitral valve prolapse, reversible or permanent sterilization for women by occluding the fallopian tubes, and for men by occluding the vas ducts or tubes, closure of septal (or other) defects in the heart or anywhere else in the body, patent foramen ovale (PFO) closure, post-biopsy tissue closure, tissue closure following minimally invasive surgical or translumninal procedures, general tissue ligation, and localized therapeutic elution. Other applications include closing an access puncture of the heart following a diagnostic or interventional procedure, such as, for example, minimally invasive, percutaneous heart valve reinforcement or replacement procedures using devices and systems such as those from Edwards Lifesciences (Irvine, Calif.).

A tissue closure system can enable the advancement/deployment of the sealing element over and/or alongside other than tubular medical devices, including tools used during medical procedure such as, for example, hemostats, cutters, tweezers, probes, biopsy devices, etc. A deployment instrument and/or sealing element can be configured to be advanced over and/or alongside additional medical devices, such as, for example, needles, hypo tubes, guide wires, electrode wires, intravenous (IV) tubes, vascular introducers, catheters, laparoscopes, endoscopes, trocars, cannulas, combination or other suitable medical devices. The disclosed systems can be packaged on or with the medical devices or tools. A deployment instrument and/or sealing element can be configured to work with medical devices of all sizes, including devices having an outer diameter of less than or equal to about 6 French, greater than or equal to about 20 French, and all sizes in between. In some embodiments, a deployment instrument and/or sealing element can be configured to work with medical devices having an outer diameter of about 6 French, 7 French, 8 French, 9 French, 10 French, 11 French, 12 French, 13 French, 14 French, 15 French, 16 French, 17 French, 18 French, 19 French or 20 French. Other suitable sizes may also be used.

In certain embodiments, a tissue closure system can be configured to operate as a stand-alone surgical system. For example, in certain embodiments a tissue closure system can be configured to operate without being advanced over or alongside or otherwise being guided by an elongate medical device.

A deployed element can be used as a temporary or permanent spacer, shun, or to displace and/or support, stabilize, reinforce, or occlude any tissue or tissues, including bone. The deployed element can be partially or completely made from many different types of materials, including, for example, a polymer, sponge, metal, metal alloy, superelastic/shape memory materials (including polymers and metallic alloys), or any other suitable material or materials. The deployed element can be deployed through a tube with a pusher element, e.g., a stylet, plunger, inner tubular member or rod, and allowed to expand before, during and/or after deployment. The deployment element can be biased in an expanded configuration. The deployed element can be maintained in a compressed configuration during positioning of the element, and allowed to expand to an expanded configuration when no longer constrained. In general, the closure device may be constrained in a smaller cross section profile, and allowed to self-expand once a constraining force is removed. In addition, the closure device may be constrained in an open position, and allowed to self-close once the opening force is removed.

The general components and/or disclosed systems, with desired modifications, can be used to temporarily or permanently close, and/or reinforce tissue access for medical procedures such as minimally invasive biopsy, other tissue removal, or diagnostic or therapeutic procedures including locations on, through, or inside the heart, locations for procedures including electrophysiology, congestive heart failure, valve related treatment (including, for example, dilation, valve reinforcement, replacement, papillary muscle treatment, chordae tendineae, and other related structures, combination and or other purposes) and/or any other locations on organs or tissue, including skin.

The systems of the present invention can facilitate less invasive surgery involving thorascopic access and visualization to the target location. In some embodiments, the systems of the invention can be suitable for use through a median stemotomy, lateral thoracotomy, intercostals port-access, mini-sternotomies, other less invasive approaches involving xiphoid access, inguinal approaches, or sub-thoracic approaches adjacent the diaphragm. In other embodiments, the systems of the present invention can be configured for catheter-based applications by elongating the shaft and altering the diameters and other feature dimensions for intravascular access.

The systems of the present invention are capable of being deployed through a thoracostomy, thoracotomy, median sternotomy, mini-sternotomy, mini-thoracotomy, xiphoid access, subthoracic access, arthroscopic, or laparoscopic approach, thereby potentially eliminating the need for long incisions to access the soft tissue and corresponding anatomic structures.

The closure devices, systems and methods can be used for temporary or permanent tissue reshaping and/or resizing. Tissues which can be reshaped and/or resized include organs, such as the stomach, lungs, etc., and other structures, such as the esophagus and structures of the heart and/or valves. For example, in certain embodiments one or more clips may alone be sufficient to reshape and/or resize tissue by one or more of accessing, gathering, pursing, bunching, cinching or holding tissue. In other embodiments, multiple clips can be connected together by a suitable tether, e.g., static or elastic, from the outer or inner surface of a tissue structure or organ. In certain embodiments, the tether can be tightened following implantation of the clips to achieve additional resizing and/or reshaping of tissue. In certain embodiments, one or more clips and/or a suitable tether can be used to resize and/or reinforce the Lower Esophageal Sphincter (LES) for gastrointestinal uses, or to resize the tissue around a heart valve.

The disclosed clips and/or delivery systems can also be configured to anchor implanted stent grafts by securing the graft to the tissue wall to prevent the graft from moving. For example, stent grafts (such as those devices and systems from W.L. Gore, Cook, Medtronic, etc.) can be used to treat an abdominal aortic aneurysm by reinforcing the aortic wall to prevent rupture. One or more clips can be deployed on the inside of the stent graft and/or on the outside of the abdominal aorta. The disclosed devices, systems and methods relating to anchoring or attachment of stent grafts, endoprosthesis, or other structures or devices, can also be used for any other locations on or inside the body.

The general closure systems can be configured to be used with robotically or computer controlled medical procedures, including surgical systems such as those available from Intuitive Surgical, Inc. (Sunnyvale, Calif.), and catheter-based technologies from Stereotaxis (St. Louis, Mo.) and Hansen Medical (Mountain View, Calif.).

The closure systems can be used to close the vessel access in larger sized catheter-based percutaneous, transluminal procedures, including heart valve reinforcement/replacement procedures, such as those from CoreValve (Irvine, Calif.), Edwards Lifesciences (Irvine, Calif.), Sadra Medical Inc. (Campbell, Calif.), etc.

II. Kits

A vessel closure system as described above can be sold to end users in the form of a kit. The kits can comprise multiple items, including but not limited to one or more deployment instruments and one or more clips. The kits can further comprise tissue cutters and tissue dilators as described above. In some embodiments, the kits can comprise swellable plugs in addition to or instead of the clips. The deployment instruments can be preloaded with the clips or plugs, or the kits can require assembly by the end user. In some embodiments, the kits can comprise an elongate medical device. In some embodiments, the kits can comprise one or more items selected from the group consisting of needles, hypo tubes, guidewires, electrode wires, intravenous wires, vascular introducers, catheters, laparoscopes, endoscopes, trocars, and cannulas. In some embodiments, the kits can comprise a compound for delivery to a tissue. The compound can be one or more of a sclerosing agent, an antibiotic, or an anti-inflammatory agent. In some embodiments, the kits can comprise one or more of any of a pair of scissors, a scalpel, a swab, a syringe, a hemostat, a lubricant, a needle, a snare, an antiseptic, or an anesthetic. Components of the kits can be designed and intended for single or multiple uses.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A guided tissue cutter having a proximal end and a distal end comprising:
(a) a surface sized and configured to receive a first cutter adapted to distally cut a target, and
(b) an elongate channel along the length of the guided tissue cutter adaptable to engage an elongate member at least one of slidably or rotationally, wherein the guided tissue cutter has one or more surfaces adapted to enable a user to handle or control operation of the cutter.

2. The guided tissue cutter of claim 1, wherein the surface sized and configured to receive a first cutter comprises a recess sized and configured to receive the first cutter.

3. The guided tissue cutter of claim 1, wherein the guided tissue cutter has a proximal end having a first width and a distal end having a second width.

4. The guided tissue cutter of claim 17 wherein the first width is greater than the second width.

5. The guided tissue cutter of claim 1 further comprising a second surface sized and configured to receive a second cutter adapted to distally cut a target further comprising a distally positioned second cutting element.

6. The guided tissue cutter of claim 1 further comprising one or more stop members adapted and configured to permit the first cutter to penetrate a predetermined region of a target tissue to a predetermined depth and further adapted and configured to inhibit further penetration of a tissue beyond the predetermined region of the target tissue by the first cutter.

7. The guided tissue cutter of claim 1, wherein the guided tissue cutter is configurable to increase a size of a tissue opening sufficiently to permit percutaneous insertion of a medical device.

8. The guided tissue cutter of claim 1, wherein the guided tissue cutter is configurable to be removable from the elongate member.

9. The guided tissue cutter of claim 1, wherein the guided tissue cutter is configurable to be removable from the elongate member by one or more of the acts of bending, clipping, cutting, snapping or tearing.

10. The guided tissue cutter of claim 1, wherein the cutter comprises one or more blades.

11. The guided tissue cutter of claim 10, wherein the one or more blades are configured to be flat, circular, diagonal or angled.

12. The guided tissue cutter of claim 10, wherein the one or more blades are adjustable to alter one or more of an incision depth and an incision width.

13. The guided tissue cutter of claim 1, wherein the cutter comprises two or more blades and the blades are configured to be any combination of one or more of flat, circular, diagonal or angled.

14. The guided tissue cutter of claim 1, wherein the cutter comprises one or more cutting elements selected from the group consisting of thermal, chemical or ultrasonic.

15. The guided tissue cutter of claim 1, wherein the elongate member comprises an elongate tubular medical device.

16. The guided tissue cutter of claim 15, wherein the elongate tubular medical device comprises a device or combination of devices selected from the group consisting of needles, hypo tubes, guidewires, electrode wires, intravenous wires, vascular introducers, catheters, laparoscopes, endoscopes, trocars, and cannulas.

17. The guided tissue cutter of claim 1, wherein the elongate member comprises an elongate non-tubular medical device.

18. The guided tissue cutter of claim 17, wherein the elongate non-tubular medical device comprises a device or combination of devices selected from the group consisting of hemostats, cutters, tweezers, probes, or biopsy devices.

19. The guided tissue cutter of claim 1, wherein the elongate member comprises a combination of one or more elongate tubular medical devices and one or more elongate non-tubular medical devices.

20. The guided tissue cutter of claim 1, wherein the cutter comprises at least one heated leading edge.

21. The guided tissue cutter of claim 20, wherein the at least one heated leading edge comprises an electrode in communication with at least one electrical conductor.

22. The guided tissue cutter of claim 20, wherein the at least one heated leading edge is configured to be heated using direct resistive heating.

23. The guided tissue cutter of claim 20, wherein the at least one heated leading edge is configured to be heated using ohmic tissue heating.

24. The guided tissue cutter of claim 1, wherein the device is configurable to cut a tissue or combination of tissues selected from the group consisting of skin, fat, ligaments, cartilage, bone, muscle and vessels.

25. A method of increasing the size of a tissue opening comprising:
(a) providing a guided tissue cutter comprising at least one surface sized and configured to receive a first cutter adapted to distally cut a target, and an elongate channel along the length of the guided tissue cutter adaptable to slidably engage an elongate member;
(b) removably attaching the guided tissue cutter to an elongate member that is percutaneously inserted into an opening in a mammalian patient;
(c) advancing the guided tissue cutter distally along the elongate member; and
(d) engaging a first cutter with a predetermined region of tissue near an opening in the mammalian patient to increase a size of the opening.

26. The method of claim 25 wherein the guided tissue cutter further comprises a stop member, the method further comprising the step of advancing the guided tissue cutter against the tissue until the stop member engages the predetermined region of tissue, the stop member permitting the first cutter to penetrate the predetermined region of tissue to a predetermined depth and thereafter inhibiting further penetration of the predetermined region of tissue.

27. The method of claim 25 further comprising the step of removably attaching the guided tissue cutter to the elongate member by clipping or snapping the guided tissue cutter onto the elongate member.

28. The method of claim 25 further comprising the step of removing the cutter from the elongate member.

29. The method of claim 25 wherein the guided tissue cutter further comprises a heatable element, the method further comprising the step of heating the element.

30. A kit for a percutaneous procedure comprising at least one of a guided tissue cutter having a proximal end and a distal end comprising: at least one surface sized and configured to receive a first cutter adapted to distally cut a target, and an elongate channel along the length of the guided tissue cutter adaptable to slidably engage an elongate member, wherein the guided tissue cutter has one or more surfaces adapted for handling or control by a user.

31. The kit of claim 30 further comprising an elongate medical device.

32. The kit of claim 30 further comprising one or more items selected from the group consisting of needles, hypo tubes, guidewires, electrode wires, intravenous wires, vascular introducers, catheters, laparoscopes, endoscopes, trocars, and cannulas.

33. The kit of claim 30 further comprising a compound for delivery to a tissue.

34. The kit of claim 33 wherein the compound is one or more of a sclerosing agent, an antibiotic, and an anti-inflammatory agent.

35. The kit of claim 30 further comprising one or more items selected from the group consisting of a pair of scissors, a scalpel, a swab, a syringe, a hemostat, a lubricant, a needle, a snare, an antiseptic, and an anesthetic.
